(19) United States
(12) Patent Application Publication
(10) Pub. No.: US 2024/0108319 A1
(43) Pub. Date: Apr. 4, 2024

(54) FECAL COLLECTION APPARATUSES, FECAL COLLECTION ATTACHMENT DEVICES, AND METHODS OF USING SAME

(71) Applicant: IDEXX Laboratories, Inc., Westbrook, ME (US)

(72) Inventors: Justin Griffin, Scarborough, ME (US); Jesse Buch, Kennebunk, ME (US); Amanda Waldron, Westbrook, ME (US); Michael P. Stone, Falmouth, ME (US)

(73) Assignee: IDEXX Laboratories, Inc., Westbrook, ME (US)

(21) Appl. No.: 18/476,618

(22) Filed: Sep. 28, 2023

Related U.S. Application Data
(60) Provisional application No. 63/411,670, filed on Sep. 30, 2022.
(60) Provisional application No. 63/476,664, filed on Dec. 22, 2022.

(51) Int. Cl. A61B 10/00; G01N 1/04

(52) U.S. Cl. CPC A61B 10/0038; G01N 1/04

(57) ABSTRACT

A fecal collection apparatus includes a main body including an insertion end and a removal end opposite the insertion end, the main body defining a main body cavity between the insertion end and the removal end, a sample collection member positionable within the main body cavity through the insertion end, the sample collection member defining a fecal engagement portion for holding a fecal sample and a handle portion spaced apart from the fecal engagement portion, and a plug provided at a removal end of the main body, the plug positionable from a closed position in which the plug restricts fluid from passing from the main body cavity through the removal end, to an open position.

### CROSS-REFERENCE To RELATED APPLICATIONS

This application claims the benefit of co-pending U.S. Provisional Patent Application Ser. No. 63/411,670, filed Sep. 30, 2022, and co-pending U.S. Provisional Patent Application Ser. No. 63/476,664, filed Dec. 22, 2022, which are hereby incorporated by reference in its entirety including the drawings.

### TECHNICAL FIELD

The present specification generally relates to sample collection apparatuses and methods for using the same, and, more specifically, to fecal collection apparatuses for collecting a fecal sample and mixing the fecal sample with an emulsifying buffer for testing.

### BACKGROUND

To perform antigen testing on a fecal sample, a user may collect a fecal sample for shipment to a testing facility. This requires the testing facility to handle the fecal sample to be tested, which may result in contamination or sample spoilage. In certain instances, the fecal sample may be collected and inserted into a syringe for testing, which eliminates the need for handling the fecal sample. However, the amount of fecal sample collected is not controlled, which may result in inaccurate results.

Accordingly, a need exists for improved sample collection apparatuses for collecting a fecal sample and permitting antigen testing of the fecal sample while reducing handling of the fecal sample during testing.

### SUMMARY

In one embodiment, a fecal collection apparatus includes: a main body including an insertion end and a removal end opposite the insertion end, the main body defining a main body cavity between the insertion end and the removal end; a sample collection member positionable within the main body cavity through the insertion end, the sample collection member defining a fecal engagement portion for holding a fecal sample and a handle portion spaced apart from the fecal engagement portion; and a plug provided at the removal end of the main body, the plug positionable from a closed position in which the plug restricts fluid from passing from the main body cavity through the removal end, to an open position.

In another embodiment, a fecal collection apparatus includes: a main body comprising an insertion end and a removal end; a plunger positioned at least partially within the main body; a sample collection member positionable within the main body through the insertion end, the sample collection member defining a fecal engagement portion for holding a fecal sample; and a guide cap positioned at the insertion end of the main body, wherein: the plunger is movable with respect to the guide cap between a locked state and an unlocked state; the plunger is inhibited from moving in an axial direction with the plunger in the locked state, the axial direction corresponding to a longitudinal axis extending from the insertion end to the removal end of the main body; and the plunger is moveable in the axial direction with the plunger in the unlocked state.

In yet another embodiment, a fecal collection attachment device is positionable onto a syringe, the fecal collection attachment device includes: a guide cap positionable onto the syringe, the guide cap defining a rail; a plunger comprising a slotted portion, the plunger rotatable relative to the guide cap to position the rail of the guide cap within the slotted portion of the plunger; and a sample collection member positionable through the plunger, the sample collection member defining a fecal engagement portion for holding a fecal sample, wherein the plunger is inhibited from moving in an axial direction relative to the guide cap when the rail of the guide cap is positioned within the slotted portion of the plunger, and wherein the plunger is permitted to move in the axial direction when the rail of the guide cap is not positioned within the slotted portion of the plunger.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a top perspective view of a fecal collection apparatus, according to one or more embodiments shown and described herein;

FIG. 2 schematically depicts a bottom perspective view of the fecal collection apparatus of FIG. 1, according to one or more embodiments shown and described herein;

FIG. 3 schematically depicts an exploded view of a brush, a main body, and a plug of the fecal collection apparatus of FIG. 1, according to one or more embodiments shown and described herein;

FIG. 4 schematically depicts an exploded view of a sample collection member, a plunger, and a guide cap of the fecal collection apparatus of FIG. 1, according to one or more embodiments shown and described herein;

FIG. 5 schematically depicts a cross-sectional view of the fecal collection apparatus taken along line 5-5 of FIG. 1, according to one or more embodiments shown and described herein;

FIG. 6 schematically depicts a top plan view of the fecal collection apparatus of FIG. 1 in the locked state, according to one or more embodiments shown and described herein;

FIG. 7 schematically depicts a top plan view of the fecal collection apparatus of FIG. 1 in the unlocked state, according to one or more embodiments shown and described herein;

FIG. 8 schematically depicts a perspective view of the fecal collection apparatus of FIG. 1 in the unlocked state and the raised position, according to one or more embodiments shown and described herein;

FIG. 9 schematically depicts a perspective view of the fecal collection apparatus of FIG. 1 in the unlocked state and the lowered position, according to one or more embodiments shown and described herein;

FIG. 10 schematically depicts an exploded cross-sectional view of a fecal collection apparatus including a sample collection member and a main body, according to one or more embodiments shown and described herein;

FIG. 11 schematically depicts a cross-sectional view of the fecal collection apparatus of FIG. 10 including a sample collection member partially inserted into a main body, according to one or more embodiments shown and described herein;

FIG. 12 schematically depicts a cross-sectional view of the sample collection member of the fecal collection apparatus of FIG. 10 in a plunging position, according to one or more embodiments shown and described herein;

FIG. 13 schematically depicts a cross-sectional view of the sample collection member of the fecal collection apparatus of FIG. 10 engaging a buffer valve in a closed position, according to one or more embodiments shown and described herein;

FIG. 14 schematically depicts a cross-sectional view of the sample collection member of the fecal collection apparatus of FIG. 10 engaging the buffer valve in an open position, according to one or more embodiments shown and described herein;

FIG. 15 schematically depicts a cross-sectional view of a buffer solution reservoir of the fecal collection apparatus of FIG. 10 in the plunging position, according to one or more embodiments shown and described herein;

FIG. 16 schematically depicts a perspective view of the fecal collection apparatus of FIG. 10 with the buffer solution reservoir in the plunging position and a plug removed from the main body of the fecal collection apparatus, according to one or more embodiments shown and described herein;

FIG. 17 schematically depicts a top perspective view of a fecal collection apparatus, according to one or more embodiments shown and described herein;

FIG. 18 schematically depicts a bottom perspective view of the fecal collection apparatus of FIG. 17, according to one or more embodiments shown and described herein;

FIG. 19 schematically depicts an exploded view of a brush, a main body, a plug, and a filter of the fecal collection apparatus of FIG. 17, according to one or more embodiments shown and described herein;

FIG. 20 schematically depicts an exploded view of a sample collection member, a plunger, a sealing member, and a guide cap of the fecal collection apparatus of FIG. 1, according to one or more embodiments shown and described herein;

FIG. 21 schematically depicts a cross-sectional view of the fecal collection apparatus taken along line 21-21 of FIG. 17, according to one or more embodiments shown and described herein;

FIG. 22 schematically depicts a partial top plan view of the fecal collection apparatus of FIG. 17 in the locked state, according to one or more embodiments shown and described herein;

FIG. 23 schematically depicts a partial top plan view of the fecal collection apparatus of FIG. 17 in the unlocked state, according to one or more embodiments shown and described herein; and

FIG. 24 schematically depicts a perspective view of the fecal collection apparatus of FIG. 17 in the unlocked state and the lowered position, according to one or more embodiments shown and described herein.

### DETAILED DESCRIPTION

Embodiments described herein are directed to fecal collection apparatuses that include a main body, a sample collection member positionable within the main body, and a plug releasably coupled to a removal end of the main body to restrict fluid from passing through the main body from removal end. The sample collection member includes a fecal engagement portion that facilitates the collection of a predetermined amount of fecal matter to be mixed with an emulsifying buffer within the main body. Various embodiments of the fecal collection apparatus and the operation of the fecal collection apparatus are described in more detail herein. Whenever possible, the same reference numerals will be used throughout the drawings to refer to the same or like parts.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, it is in no way intended that an order or orientation be inferred. This holds for any possible non-express basis for interpretation, for example and without limitation: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include one or more referents unless the context clearly dictates otherwise. Thus, for example, reference to “a” component includes aspects having a single component or multiple components, unless the context clearly indicates otherwise.

As will be appreciated, the fecal collection apparatus disclosed herein permits a user to collect a fecal sample and store the fecal sample in a main body, such as a syringe, which may be used to mix the fecal sample with an emulsifying buffer and dispense a particular amount of the mixture. This eliminates the need for transferring the fecal sample to another dispensing mechanism, which may result in contamination of the fecal sample.

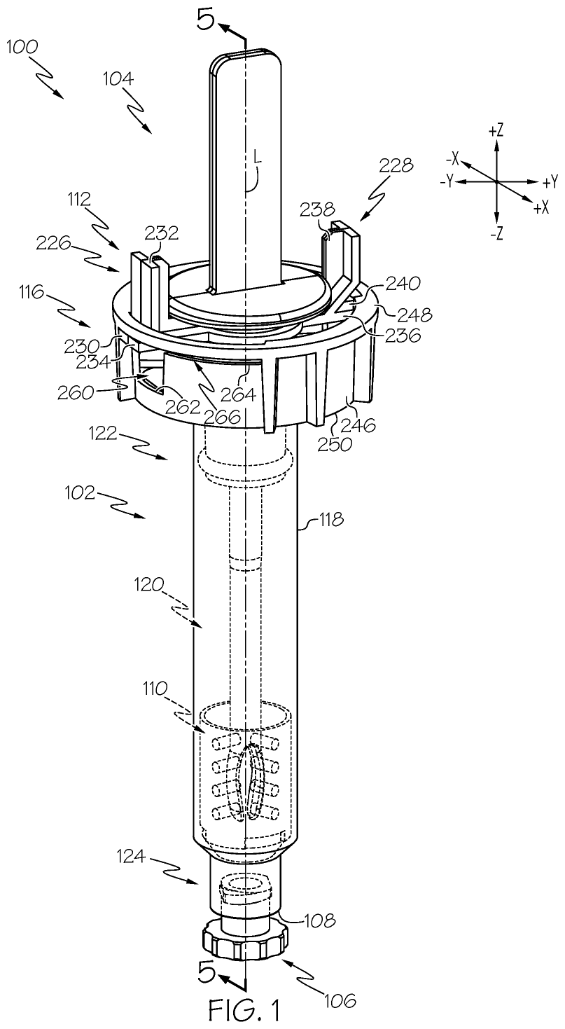

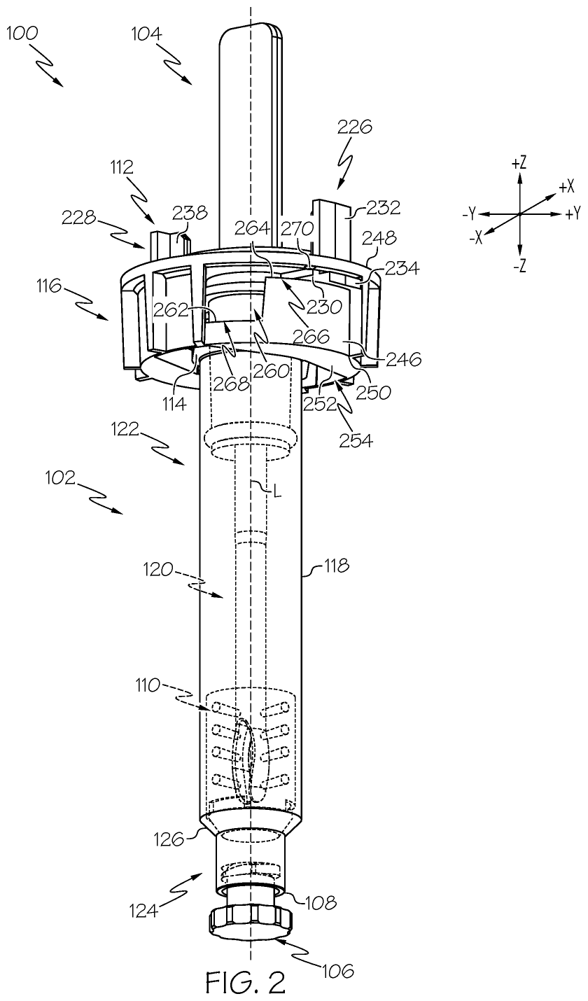

Referring now to FIGS. 1 and 2, a fecal collection apparatus 100 is illustrated according to one or more embodiments described herein. In embodiments, the fecal collection apparatus 100 generally includes a main body 102, a sample collection member 104 for collecting a fecal sample and positionable within the main body 102, and a plug 106 releasably coupled to a removal end 108 of the main body 102. It should be appreciated that an emulsifying buffer, agent, or solution may be initially provided within the main body 102, and the plug 106 prevents the emulsifying buffer from being dispensed out of the removal end 108 of the main body 102. In some embodiments, a brush 110 is provided within the main body 102 and facilitates scraping of the sample collection member 104 to remove the fecal sample from the sample collection member 104 and mix the fecal sample with the emulsifying buffer.

In embodiments, the fecal collection apparatus 100 includes a plunger 112 positioned at least partially within the main body 102 through an insertion end 114 of the main body 102 opposite the removal end 108. The sample collection member 104 is insertable through the plunger 112 and into the main body 102. In some embodiments, the fecal collection apparatus 100 includes a guide cap 116 positioned at the insertion end 114 of the main body 102. The guide cap 116, in embodiments, is engaged with the plunger 112. As described in more detail herein, in embodiments, the guide cap 116 is rotatably secured to the main body 102. In embodiments, the plunger 112 is rotatable relative to the guide cap 116 so as to permit the plunger 112 to be positionable between an unlocked state and a locked state. In the unlocked state, the sample collection member 104 and the plunger 112 are permitted to be inserted further into the main body 102 by application of a force, as shown in FIG. 1. In the locked state the sample collection member 104 and the plunger 112 are prevented from being further inserted into the main body 102 by application of a force, as shown in FIG. 2. With the plug 106 removed from the removal end 108 of the main body 102 and the plunger 112 positioned into the unlocked state, movement of the sample collection member 104 further into the main body 102 presses the plunger 112 further into the main body 102. As the plunger 112 moves into the main body, the emulsifying buffer mixed with the fecal sample passes out of the main body 102 through the removal end 108. In some embodiments, the main body 102 is a syringe, and more particularly, a syringe body. Moreover, the guide cap 116, the plunger 112, and the sample collection member 104 may define a fecal collection attachment device positionable as a retrofit onto a syringe body, such as the main body 102.

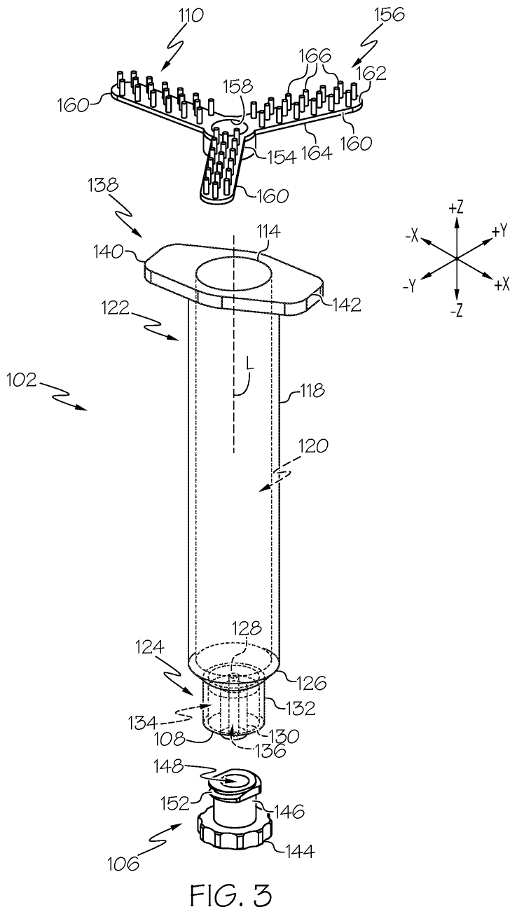

Referring now to FIGS. 3, an exploded view of the main body 102, the plug 106, and the brush 110 are shown in greater detail. As described herein, the plug 106 is positionable at the removal end 108 of the main body 102 to prevent emulsifying buffer from flowing out of the removal end 108 of the main body 102. The brush 110 (shown in an expanded or unfolded position in FIG. 3) is positionable within the main body 102.

With respect to the main body 102, the main body 102 includes a main body wall 118 defining a main body cavity 120. The main body wall 118 includes the removal end 108 provided at a lowermost point of the main body 102 and the insertion end 114 provided at an uppermost point of the main body 102 opposite the removal end 108. The main body 102 further defines a longitudinal axis L extending in the +/−Z direction of the coordinate axes depicted in the drawings.

In embodiments, the main body 102 includes an upper body portion 122, which includes the insertion end 114, and a lower body portion 124, which includes the removal end 108. In embodiments (not depicted), an outer diameter of the lower body portion 124 is equal to or greater than an outer diameter of the upper body portion 122. In other embodiments, as shown, the outer diameter of the lower body portion 124 is less than the outer diameter of the upper body portion 122. In embodiments in which the outer diameter of the lower body portion 124 is less than the outer diameter of the upper body portion 122, an arcuate or tapered wall portion 126 may be provided to extend from the upper body portion 122 to the lower body portion 124. In embodiments, the lower body portion 124 includes a base wall 128, an inner longitudinal wall 130 extending from the base wall 128, and an outer longitudinal wall 132 extending from the base wall 128 and spaced apart from the inner longitudinal wall 130. The outer longitudinal wall 132 has an inner diameter greater than an outer diameter of the inner longitudinal wall 130 such that a recess 134 is defined between the outer longitudinal wall 132 and the inner longitudinal wall 130. The recess is closed off by the base wall 128 at an end thereof opposite the removal end 108. A conduit 136 is defined extending through the base wall 128 and the inner longitudinal wall 130 to be in fluid communication with the main body cavity 120. Thus, the emulsifying buffer is permitted to flow out of the removal end 108 of the main body 102 from the main body cavity 120 of the upper body portion 122 through the conduit 136 defined in the inner longitudinal wall 130 of the lower body portion 124.

In some embodiments, the main body 102 includes a flange 138 provided at the insertion end 114 thereof and extending radially relative to the longitudinal axis L of the main body 102. That is, the flange 138 extends in a direction generally perpendicular to the longitudinal axis L of the main body 102. In embodiments, the flange 138 is provided at the insertion end 114 of the main body 102 and includes a first flange portion 140 extending in a first radial direction relative to the longitudinal axis L from the insertion end 114 of the main body 102 and a second flange portion 142 extending in a second radial direction from the insertion end 114 of the main body 102. In embodiments, the first flange portion 140 and the second flange portion 142 are diametrically opposed to one another and extend in opposite radial directions, however this is merely an example. Although the flange 138 depicted herein includes a pair of flange portions 140, 142, it should be appreciated that any number of flange portions may be provided. For example, the flange 138 may include a single flange portion or more than two flange portions. In embodiments, in which more than two flange portions are provided, the flange portions may be spaced apart from one another in any suitable arrangement such as, for example, equidistantly from one another in a circumferential direction.

Referring still to FIG. 3, the plug 106 is shown removed from the main body 102. In the embodiment depicted in FIG. 3, the plug 106 includes a gripping portion 144 and an extension portion 146 extending from the gripping portion 144. The extension portion 146, in embodiments, defines a cavity 148 therein, extending toward the gripping portion 144.

In some embodiments, the plug 106 includes an exterior thread 152 formed at an end of the extension portion 146, which threadedly engages an interior thread formed on the outer longitudinal wall 132 of the lower body portion 124 to retain the plug 106 within the lower body portion 124. In some embodiments, the plug 106 may include an interior thread formed at an end of the extension portion 146 which threadedly engages an exterior thread formed on the inner longitudinal wall 130 of the lower body portion 124 to retain the plug 106 within the lower body portion 124.

While in the embodiment depicted in FIG. 3, the plug 106 is shown as a component threadedly engageable with the lower body portion 125 of the main body 102, it should be understood that this is merely an example. For example, in some embodiments, the plug 106 may be coupled to the lower body portion 125 of the main body 102 via a frangible seal. For example, the main body 102 and the plug 106, in embodiments, are a one piece, monolithic structure and the plug 106 may be removed from the main body 102 by applying force in a direction away from the removal end 108 of the main body 102.

In some embodiments, the plug 106 may be press-fitted within the removal end 108 of the main body 102. Regardless of the specific physical implementation of the connection, it should be understood that the plug 106 is removable from the lower body portion 124 of the main body 102 by applying a force against the plug 106.

Referring still to FIG. 3, the brush 110 is illustrated separate from the main body 102. The brush 110 is positionable between an unfolded position when not positioned within the main body 102, as shown in FIG. 3, and a folded position when positioned within the main body 102, as shown in FIGS. 1 and 2. The brush 110 includes a base 154 and a cleaning member 156 provided on the base 154. However, it should be appreciated that the cleaning member 156 may extend from the base 154 as a one piece, monolithic structure as opposed to a separate structure provided on the base 154. An aperture 158 extends through the brush 110 to allow the mixture of emulsifying buffer and fecal sample to pass through the brush 110 when the cleaning member 156 is positioned within the main body 102. The cleaning member 156 includes one or more cleaning portions 160 extending in a radial direction from the base 154 when the cleaning member 156 is in the unfolded position. In embodiments, the cleaning portions 160 may include apertures to allow a fluid to pass through.

As shown in FIG. 3, the cleaning member 156 includes three cleaning portions 160 extending outwardly from the base 154 with the cleaning member 156 in the unfolded position. The cleaning portions 160 may be equidistantly spaced apart from the one another in a circumferential direction with respect to the base 154, however this is merely an example. While the embodiment depicted in FIG. 3 shows three cleaning portions 160, it should be appreciated that the cleaning member 156 may include any suitable number of cleaning portions 160 such as, for example, one, two, or more than three cleaning portions 160. Each cleaning portion 160 has an inner surface 162 and an outer surface 164 opposite the inner surface 162. In the unfolded position, the outer surface 164 faces outwardly toward the base 154, and the inner surface 163 faces inwardly. In embodiments, the inner surface 162 of each cleaning portion 160 includes a plurality of bristles 166 extending in a direction opposite the base 154 in the unfolded position. The bristles 166 may be provided in any suitable arrangement along the inner surface 162 of the cleaning portions 160. For example, the bristles 166 may be arranged in one or more rows. In embodiments, the cleaning portions 160 may be a deformable or flexible members, and may be formed of a flexible plastic, rubber, or the like, so as to allow the cleaning portions 160 of the cleaning member 156 to fold relative to the base 154 and position the brush 110 into the folded position. In other embodiments, one or more hinge portions may be provided joining the cleaning portions 160 to a location of the cleaning member 156 adjacent the aperture 158 formed in the brush 110 so as to permit the cleaning portions 160 to bend relative to the base 154 at a respective hinge portion, such as a living hinge formed at the intersection of the cleaning portions 160 with the base 154. In embodiments in which one or more hinge portions are provided, the cleaning portions 160 may be formed from a rigid or substantially rigid material while still permitting the brush 110 to fold between the unfolded position and the folded position. In embodiments, the bristles 166 are formed from a flexible material providing brushing and cleaning ability.

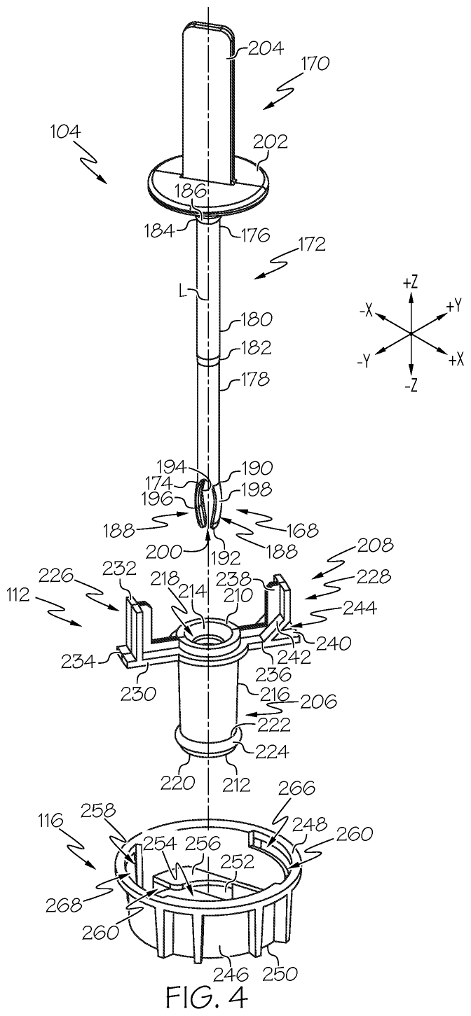

Referring now to FIG. 4, an exploded view of the sample collection member 104, the plunger 112, and the guide cap 116 are shown in greater detail. As described in detail herein, the sample collection member 104, the plunger 112, and the guide cap 116 are positioned at the insertion end 114 of the main body 102.

With respect to the sample collection member 104, the sample collection member 104 defines a fecal engagement portion 168 for collecting and retaining a fecal sample and a handle portion 170 spaced apart from the fecal engagement portion 168. In embodiments, the sample collection member 104 includes an elongated member 172 having a fecal engagement end 174 and a handle end 176 opposite the fecal engagement end 174. The fecal engagement portion 168 is provided at the fecal engagement end 174 of the elongated member 172 and the handle portion 170 is provided at the handle end 176 of the elongated member 172. In embodiments, the fecal engagement portion 168, the handle portion 170, and the elongated member 172 are formed as a one-piece, monolithic structure. In embodiments, the elongated member 172 includes a first elongated member portion 178 proximate the fecal engagement portion 168, a second elongated member portion 180 proximate the handle portion 170, and a tapered portion 182 extending between the first elongated member portion 178 and the second elongated member portion 180. Specifically, the tapered portion 182 extends from the second elongated member portion 180 to the first elongated member portion 178 such that a diameter of the first elongated member portion 178 is less than a diameter of the second elongated member portion 180. In some embodiments, a groove 184 is formed in the elongated member 172 proximate the handle end 176 of the elongated member 172. In embodiments, a sealing member 186, such as an O-ring, is provided within the groove 184 for providing a fluid tight seal with the plunger 112.

The fecal engagement portion 168 is deformable between an uncompressed position and a compressed position. The fecal engagement portion 168 includes one or more pincers 188 extending from the fecal engagement end 174 of the elongated member 172. As shown in FIG. 4, the fecal engagement portion 168 includes two pincers 188 spaced apart from one another. However, it should be appreciated that the fecal engagement portion 168 may include any suitable number of pincers 188 for collecting a fecal sample therebetween. For example, the fecal engagement portion 168 may include one pincer 188 for collecting a fecal sample. In other embodiments, the fecal engagement portion 168 may include three or more pincers 188. Each pincer 188 includes a proximal end 190 extending from the elongated member 172, a distal end 192 opposite the proximal end 190, an inner surface 194, and an outer surface 196 opposite the inner surface 194. In embodiments, the pincers 188 may be spaced apart from one another to define a receptacle 200 in which the fecal sample is retained. In embodiments, each pincer 188 may have an arcuate shape such that a distance between opposite pincers 188 decreases further along a length of the pincers 188 in a direction opposite elongated member 172 (i.e., in the −Z-direction as depicted). In this embodiment, each pincer 188 includes a widened point 198 formed at a substantial medial location between the proximal end 190 and the distal end 192 of the pincer 188. As such, the receptacle 200 defined by the pincers 188 may have a substantially elliptical geometry extending from the proximal end 190 and the distal end 192 of the pincers 188. The fecal engagement portion 168 may be formed from a deformable or flexible material, such as a flexible plastic, rubber, or the like, to permit the pincers 188 to compress toward one another upon application of a force in a direction toward the receptacle 200 from the outer surface 196 of the pincers 188. In other embodiments, the fecal engagement portion 168 may be formed from a substantially rigid material, but flexibly joined to the elongated member 172 to allow the pincers 188, and more particularly the distal end 192 of the pincers 188, to compress toward one another upon application of a force.

With the fecal engagement portion 168 in the uncompressed position, the fecal engagement portion 168 defines an uncompressed sample collection outer diameter between the outer surface 196 of each pincer 188 and at the widened point 198 of each pincer 188. With the fecal engagement portion 168 in the compressed position, such as when the fecal engagement portion 168 is being inserted through the plunger 112, the fecal engagement portion 168 defines a compressed sample collection outer diameter between the outer surface 196 of each pincer 188 and at the widened point 198 of each pincer 188 that is less than the uncompressed sample collection outer diameter when the fecal engagement portion 168 is in the uncompressed position.

The handle portion 170 is provided at the handle end 176 of the elongated member 172 opposite the fecal engagement portion 168. The handle portion 170 includes a base plate 202 provided at the handle end 176 of the elongated member 172 and a tab 204 extending from the base plate 202 in a direction opposite the elongated member 172. In embodiments, as shown, the base plate 202 has a circular shape. However, it should be appreciated that the base plate 202 may have any suitable shape.

Referring still to FIG. 4, the plunger 112 includes a plunger body 206 and a plunger guide 208. The plunger body 206 has an upper end 210, a lower end 212 opposite the upper end 210, an inner surface 214, and an outer surface 216 opposite the inner surface 214 extending between the upper end 210 and the lower end 212. In embodiments, the inner surface 214 and the outer surface 216 are each tapered from the upper end 210 toward the lower end 212. The inner surface 214 of the plunger body 206 defines an open-ended channel 218 and an end of the channel 218 opposite the plunger guide 208 defines a sample collection aperture 220. The sample collection aperture 220 defines an aperture inner diameter. In embodiments, a groove 222 is defined in the outer surface 216 of the plunger body 206 proximate the lower end 212 of the plunger body 206 and a sealing member 224, such as an O-ring, gasket, or the like, is provided within the groove 222 to provide a fluid tight seal between the outer surface 216 of the plunger body 206 and the main body 102.

Referring still to FIG. 4, the plunger guide 208 is provided at the upper end 210 of the plunger body 206. The plunger guide 208 includes a first finger 226 extending outwardly from the outer surface 216 of the plunger body 206. In embodiments, as shown, the plunger guide 208 includes the first finger 226 and a second finger 228 extending outwardly from the outer surface 216 of the plunger body 206. In embodiments, the first finger 226 and the second finger 228 are diametrically opposed to one another such that the first finger 226 and the second finger 228 are positioned on opposite sides of the plunger body 206, however it should be understood that this is merely an example. Although the plunger guide 208 is illustrated as including the first finger 226 and the second finger 228, it should be appreciated that the plunger guide 208 may include any number of fingers, such as one finger or more than two fingers.

With respect to the first finger 226, the first finger 226 includes a lower finger portion 230 and an upright finger portion 232 extending from the lower finger portion 230. The lower finger portion 230 defines an extension 234 extending in a direction opposite the plunger body 206 from a point at which the upright finger portion 232 extends from the lower finger portion 230. Similarly, the second finger 228 includes a lower finger portion 236 and an upright finger portion 238 extending from the lower finger portion. The lower finger portion 236 of the second finger 228 similarly defines an extension 240 extending from a point at which the upright finger portion 238 extends from the lower finger portion 236. As shown in FIG. 4, the upright finger portion 238 of the second finger 228 defines a tapered portion 242. The tapered portion 242 forms a recess 244 with the lower finger portion 236 such that a length of the extension 240 of the second finger 228, which extends from the upright finger portion 238 of the second finger 228, is greater than a length of the extension 234 of the first finger 226, which extends from the upright finger portion 232 of the first finger 226.

Referring still to FIG. 4, the guide cap 116 includes an outer wall 246 including an upper end 248 and a lower end 250. A bottom wall 252 is provided at the lower end 250 of the outer wall 246 and a cutout 254 is defined in the bottom wall 252. As described in more detail herein, the cutout 254 permits the guide cap 116 to be positioned on the main body 102 (FIG. 3) at the insertion end 114 by permitting the flange 138 (FIG. 3) of the main body 102 to extend through the cutout 254 and be received within the guide cap 116. The guide cap 116 includes a blocking wall 256 extending toward a center of the guide cap 116 from the outer wall 246. Although only one blocking wall 256 is shown, it should be appreciated that a blocking wall 256 may extend from opposite sides of the outer wall 246 of the guide cap 116. A lower retaining groove 258 is defined by an overlap between the bottom wall 252 and the blocking wall 256. As described in more detail herein, the flange 138 (FIG. 3) of the main body 102 is insertable through the cutout 254 formed in the bottom wall 252 and positionable within the lower retaining groove 258 when the guide cap 116 is rotated relative to the main body 102 so as to prohibit movement of the main body 102 independent of the guide cap 116 along the longitudinal axis L, thereby retaining the guide cap 116 on the insertion end 114 of the main body 102.

Referring now to FIGS. 1, 2, and 4, the guide cap 116 includes an upper retaining groove 260 defined in the outer wall 246. The upper retaining groove 260 is defined by a lower recess wall 262 and an upper recess wall 264 defined in the outer wall 246 of the guide cap 116. More specifically, the upper retaining groove 260 defines a first groove portion 266 formed between the upper recess wall 264 and the upper end 248 of the guide cap 116, and a second groove portion 268 formed between the lower recess wall 262 and the upper end 248 of the guide cap 116. As best illustrated in FIG. 2, the first groove portion 266 has a height defined by a distance between the upper end 248 of the guide cap 116 and the upper recess wall 264 that is greater than a height of the second groove portion 268 defined by a distance between the upper end 248 of the guide cap 116 and the lower recess wall 262. It should be appreciated that the height of the first groove portion 266 is only marginally greater than a thickness of the lower finger portion 230 of the first finger 226 to prevent substantial movement of the plunger 112 along the longitudinal axis L. In embodiments, the height of the first groove portion 266 is between 5% and 50% greater than the thickness of the lower finger portion 230. In embodiments, the height of the first groove portion 266 is between 5% and 20% greater than the thickness of the lower finger portion 230. As described in more detail herein, the upper retaining groove 260 partially receives the first finger 226 of the plunger 112 and, more specifically, the first finger 226 is positionable between the first groove portion 266 or the second groove portion 268 based on rotation of the plunger 112 relative to the guide cap 116 with the guide cap 116 positioned on the main body 102. As shown in FIGS. 2 and 4, the guide cap 116 includes a pair of upper retaining grooves 260 formed therein such that each upper retaining groove 260 receives a corresponding one of the first finger 226 or the second finger 228. In embodiments, as shown in FIG. 2, a projection 270 extends from the upper end 248 of the outer wall 246 within the upper retaining groove 260 and toward the upper recess wall 264. In some embodiments, a projection 270 extends from the upper end 248 of the outer wall 246 within each upper retaining groove 260. In other embodiments, the projection 270 extends from the upper recess wall 264 to the upper end 248 of the outer wall 246. In other embodiments, a projection 270 is provided on both the upper recess wall 264 and the upper end 248 of the outer wall 246 within each upper retaining groove 260.

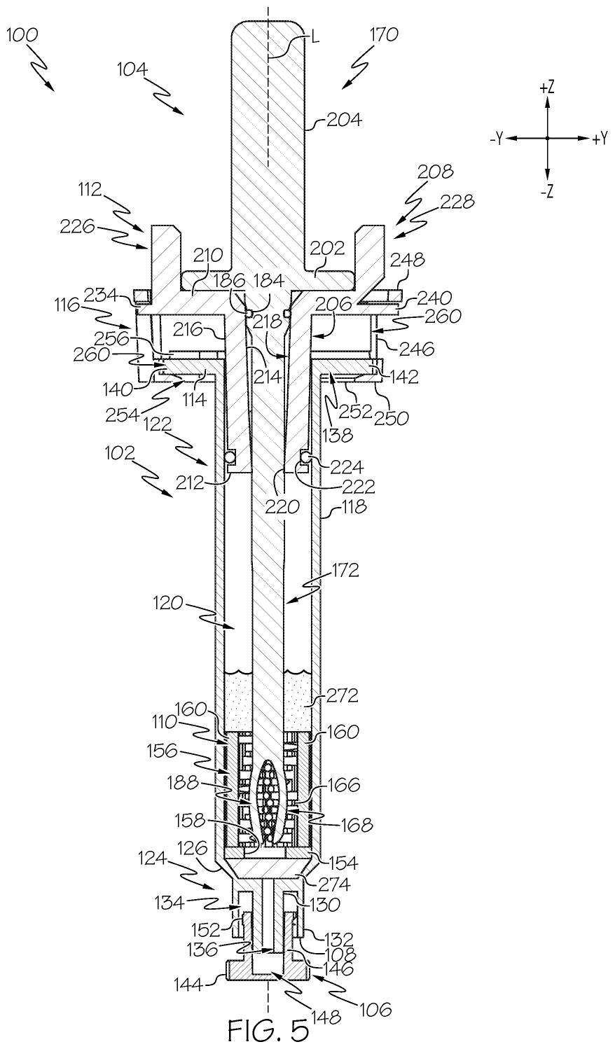

Referring now to FIG. 5, a cross-section view of the fecal collection apparatus 100 is shown in an assembled state. As shown, in the assembled state, the plug 106 positioned at the removal end 108 of the main body 102. In the embodiment depicted in FIG. 5, the extension portion 146 of the plug 106 is received within the recess 134 formed between the inner longitudinal wall 130 and the outer longitudinal wall 132 of the main body 102. Further, the inner longitudinal wall 130 of the main body 102 is received within the cavity 148 formed in the extension portion 146 of the plug 106. As described herein, the plug 106 may be press fitted into the removal end 108 of the main body 102, integrally formed therewith as a one piece, monolithic structure and detachable from the main body 102 at the frangible seal, or threadedly engaged with the main body 102 by the exterior thread 152. With the extension portion 146 of the plug 106 inserted into the recess 134 of the lower body portion 124, the gripping portion 144 remains positioned exteriorly of the lower body portion 124 so that the gripping portion 144 may remain accessible by a user and the plug 106 can be removed from the main body 102 upon application of force. As shown, the emulsifying buffer 272 is provided into the main body cavity 120 and retained therein by the plug 106, which prevents the emulsifying buffer 272 from exiting the main body 102 through the conduit 136 of the lower body portion 124.

As shown in FIG. 5, the brush 110 is positioned within the main body cavity 120. In the embodiment shown in FIG. 5, the brush 110 is positioned proximate the removal end 108 of the main body 102. The brush 110 is shown in the folded position within the main body cavity 120 such that the cleaning portions 160 extend substantially along the longitudinal axis L of the main body 102 such that the bristles 166 are directed inwardly. In addition, in the configuration show in FIG. 5, the brush 110 is positioned such that the aperture 158 extending through the base 154 of the brush 110 is coaxially aligned with the conduit 136 formed in the lower body portion 124 of the main body 102.

In embodiments, a filter 274 may be positioned within the main body cavity 120 between the base 154 of the brush 110 and the base wall 128 of the lower body portion 124. The filter 274 may be formed from a porous material or have a plurality of channels formed therein to permit particles having a diameter equal to or less than a predetermined diameter mixed with the emulsifying buffer 272 to pass through the filter 274 and toward the removal end 108 of the main body 102. As such, particles having a diameter greater than the predetermined diameter defined by the filter 274 remain on the same side of the filter 274 as the brush 110.

In use, the guide cap 116 is positioned onto the insertion end 114 of the main body 102 by inserting the insertion end 114, particularly the flange 138 of the main body 102, through the cutout 254 of the bottom wall 252 of the guide cap 116 and rotating the guide cap 116 relative to the main body 102 such that the flange 138 is retained within the lower retaining groove 258 of the guide cap 116. Specifically, once the guide cap 116 is rotated relative to the main body 102, the first flange portion 140 and the second flange portion 142 are each positioned within a respective lower retaining groove 258 formed between the blocking walls 256 and the bottom wall 252 of the guide cap 116. Thus, the guide cap 116 is prohibited from moving along the longitudinal axis L of the main body 102.

Referring still to FIG. 5, the plunger 112 is shown inserted into the insertion end 114 of the main body 102 and engaged with the guide cap 116. In embodiments, the plunger 112 may be initially positioned within the guide cap 116, and the guide cap 116 subsequently attached to the main body 102. However, it should be appreciated that, in embodiments, it may be possible to position the plunger 112 into the insertion end 114 of the main body 102 and engage the guide cap 116 after the guide cap 116 is already secured onto the insertion end 114 of the main body 102. In some embodiments, the guide cap 116 may be formed as a one piece, monolithic structure with the main body 102 to eliminate the need to couple the guide cap 116 onto the main body 102.

As shown in FIG. 5, the first finger 226 and the second finger 228 of the plunger 112 are positioned to extend through the upper retaining grooves 260 of the guide cap 116. With the plunger body 206 positioned into the main body cavity 120, the sealing member 224 circumscribing the outer surface 196 of the plunger body 206 proximate the lower end 212 thereof provides a fluid tight seal between the outer surface 196 of the plunger body 206 and the main body wall 118 of the main body 102. Initially, as shown, the first finger 226 and the second finger 228 are each positioned within the first groove portion 266 of a corresponding upper retaining groove 260 such that the plunger 112 is in the locked state. As described herein, when the plunger 112 is in the locked state, the plunger 112 is restricted from moving along the longitudinal axis L of the main body 102 in a direction toward the insertion end 114 of the main body 102.

Thereafter, the sample collection member 104 may be utilized to collect a fecal sample, such as by inserting the fecal engagement portion 168 into a fecal matter, and collecting the fecal sample within the receptacle 200 between the pincers 188. The sample collection member 104 may then be inserted into the main body 102 through the sample collection aperture 220 formed in the plunger body 206 and through the insertion end 114 of the main body 102 into the main body cavity 120. Prior to inserting the sample collection member 104 into the plunger 112, the fecal engagement portion 168 is in the uncompressed position with the pincers 188 spaced apart from one another to define the uncompressed sample collection outer diameter. As described herein, the uncompressed sample collection outer diameter of the plunger body 206 is greater than the aperture inner diameter of the sample collection aperture 220. Therefore, as the fecal engagement portion 168 is inserted into the plunger 112 and contacts the inner surface 214 of the plunger body 206 at the sample collection aperture 220, the inner surface 214 of the plunger body 206 compresses the pincers 188 of the fecal engagement portion 168 to position the fecal engagement portion 168 into the compressed position. As described herein, when the fecal engagement portion 168 is in the compressed position, the fecal engagement portion 168 has a compressed sample collection outer diameter that is less than the aperture inner diameter. Thus, the fecal engagement portion 168 is permitted to pass through the sample collection aperture 220. In doing so, excess fecal sample clinging to an outside of the fecal engagement portion 168 is retained within the plunger 112, rather than being further inserted into the main body cavity 120 and mixing with the emulsifying buffer 272. This ensures that only a controlled amount of fecal matter is permitted to mix with the emulsifying buffer 272 within the main body cavity 120. As shown, once the sample collection member 104 is fully inserted into the main body 102, the sealing member 186 on the elongated member 172 of the sample collection member 104 forms a fluid tight seal between the elongated member 172 of the sample collection member 104 and the inner surface 214 of the plunger body 206. Thus, the emulsifying buffer 272 is prevented from flowing through the channel 218 formed in the plunger 112 and out of the insertion end 114 of the main body 102.

To mix the fecal sample with the emulsifying buffer 272, the sample collection member 104 is moved in opposite directions along the longitudinal axis L of the main body 102 throughout the main body cavity 120 and rotated. In doing so, the bristles 166 of the brush 110 scrape the fecal sample out of the fecal engagement portion 168 of the sample collection member 104 so that the fecal sample mixes with the emulsifying buffer 272.

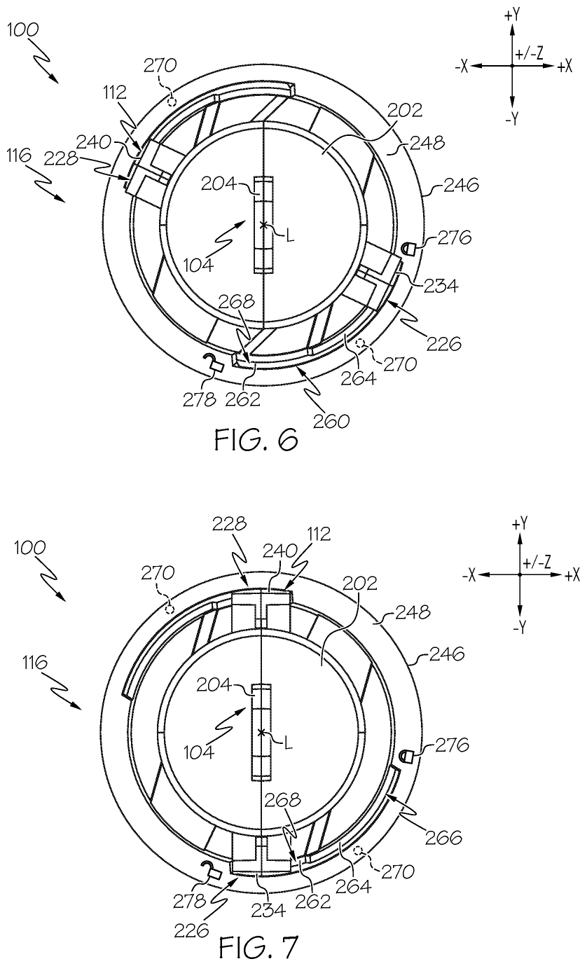

Referring now to FIGS. 6 and 7, the plunger 112 is shown in the locked state and the unlocked state, respectively. When in the locked state, as shown in FIG. 6, the first finger 226 and the second finger 228 of the plunger 112 are positioned within the first groove portion 266 of a corresponding upper retaining groove 260. More specifically, when the plunger 112 is in the locked state, the first finger 226 and the second finger 228 are positioned between the upper end 248 of the outer wall 246 and the upper recess wall 264 of a corresponding upper retaining groove 260. The plunger 112 is prevented from inadvertently moving from the locked state to the unlocked state due to the projection 270 extending from the upper end 248 of the outer wall 246 above the upper recess wall 264. Specifically, when the plunger 112 is in the locked state, the projection 270 adds a predetermined amount of resistance to the plunger 112 rotating toward the unlocked state as the projection 270 abuts against the first finger 226. The specific amount of resistance corresponds to the size of the projection 270 extending into the upper retaining groove 260. Upon a predetermined force being applied to rotate the plunger 112 from the locked state to the unlocked state, the first finger 226 is permitted to pass across the projection 270 and be positioned into the second groove portion 268. Similarly, upon a predetermined force being applied to rotate the plunger 112 back to the unlocked state from the locked state, the first finger 226 passes back across the projection 270 and is positioned into the first groove portion 266.

In the unlocked state, as shown in FIG. 7, the first finger 226 and the second finger 228 of the plunger 112 are positioned within the second groove portion 268 of a corresponding upper retaining groove 260. More specifically, when the plunger 112 is in the locked state, the first finger 226 and the second finger 228 are positioned between the upper end 248 of the outer wall 246 and the lower recess wall 262 of a corresponding upper retaining groove 260. As the height of the second groove portion 268 extending between the upper end 248 of the outer wall 246 and the lower recess wall 262 is significantly greater than a thickness of the first finger 226 and the second finger 228, the plunger 112 is permitted to move toward the removal end of the main body 102 along the longitudinal axis L of the main body 102 until a point at which the first finger 226 and the second finger 228 abut against the lower recess wall 262.

In embodiments, indicia may be provided on the upper end 248 of the outer wall 246 of the guide cap 116 to provide a visual indication of a position or state of the plunger 112. The indicia may include a lock symbol 276 to indicate that the plunger 112 is in the locked state and an unlock symbol 278 to indicate that the plunger 112 is in the unlocked state. For example, when the plunger 112 is in the locked state, as shown in FIG. 6, the first finger 226 is provided within the first groove portion 266 of the upper retaining groove 260 and positioned proximate the lock symbol 276. Alternatively, when the plunger 112 is in the unlocked state, as shown in FIG. 7, the first finger 226 is provided within the second groove portion 268 of the upper retaining groove 260 and positioned proximate the unlock symbol 278.

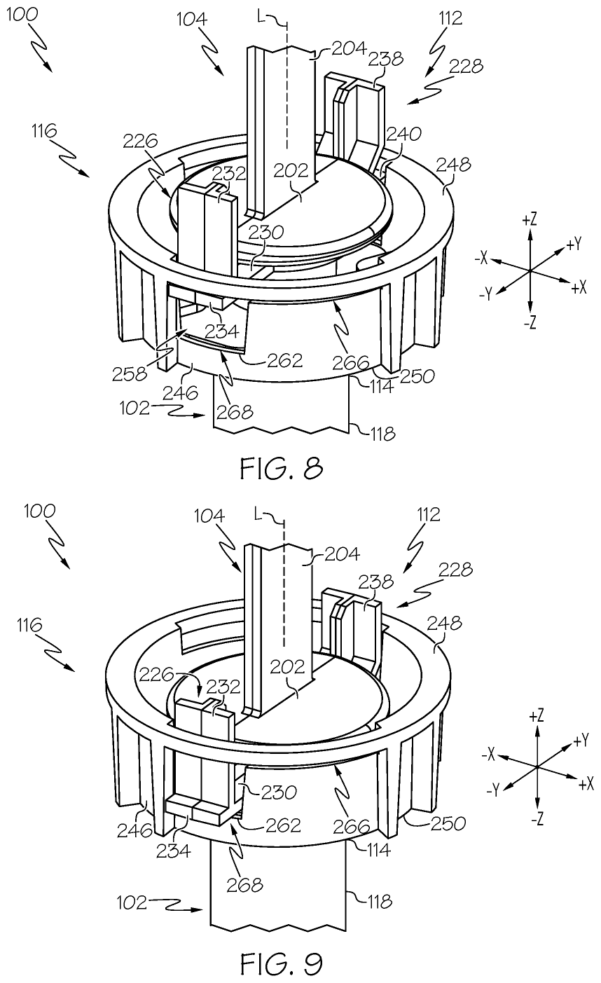

Referring now to FIG. 8, the plunger 112 is shown in the unlocked state with the first finger 226 provided within the second groove portion 268 of the upper retaining groove 260. More particularly, the plunger 112 is shown in a raised position abutting against the upper end 248 of the outer wall 246 of the plunger 112. When in the raised position, it should be appreciated that the mixture of emulsifying buffer 272 (FIG. 5) and fecal sample is not being dispensed out of the main body 102. To dispense the mixture of emulsifying buffer 272 and fecal sample out of the removal end 108 of the main body 102, the sample collection member 104 is pressed in a direction toward the removal end 108 of the main body 102 along the longitudinal axis L such that the handle portion 170 of the sample collection member 104 presses against the plunger 112 and the plunger 112 moves toward the removal end 108 of the main body 102 along the longitudinal axis L. Accordingly, as shown in FIG. 9, the plunger 112 is shown in a lowered position such that the plunger 112 has traveled a predetermined stroke and the first finger 226 abuts against the lower recess wall 262. In use, the sample collection member 104 and the plunger 112 may alternate between the raised position and the lowered position to dispense an additional supply of mixture of the emulsifying buffer 272 and the fecal sample.

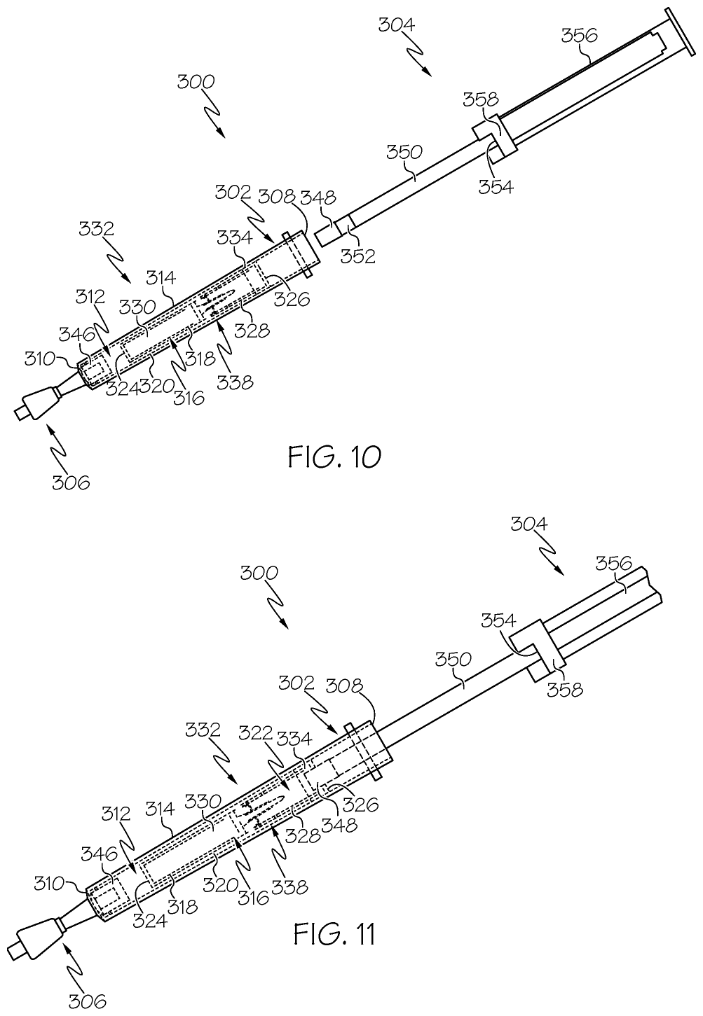

Referring now to FIGS. 10-16, another embodiment of a fecal collection apparatus 300 is illustrated according to one or more embodiments described herein. It should be appreciated that the fecal collection apparatus 300 may include features of the fecal collection apparatus 100 (FIGS. 1-9) discussed herein such as, for example, the brush 110, the plunger 112 and the guide cap 116, without deviating from the scope of the present disclosure. In embodiments, the fecal collection apparatus 300 generally includes a main body 302, a sample collection member 304 for collecting a fecal sample and positionable within the main body 302, and a plug 306 releasably coupled to the main body 302.

Referring to FIG. 10, the main body 302 includes an insertion end 308 and a removal end 310 opposite the insertion end 308. The main body 302 defines a main body cavity 312 between the insertion end 308 and the removal end 310. More particularly, the main body 302 includes a main body wall 314 defining the main body cavity 312. The main body wall 314 includes the removal end 310 provided at a lowermost point of the main body 302 and the insertion end 308 provided at an uppermost point of the main body 302 opposite the removal end 310. It should be appreciated that the main body 302 may include similar structure to that of the main body 102 described hereinabove.

The main body 302, in embodiments, includes a buffer solution reservoir 316 provided within the main body cavity 312. The buffer solution reservoir 316 includes a reservoir body 318 including one or more sidewalls 320 defining a reservoir cavity 322. The reservoir cavity 322 has a first end 324 facing the removal end 310 of the main body 302 and a second end 326 facing the insertion end 308 of the main body 302 opposite the first end 324. The first end 324 of the reservoir body 318 is a closed end and the second end 326 of the reservoir body 318 is an open end to permit the sample collection member 304 to be insertable into the reservoir cavity 322. The reservoir body 318 further defines one or more openings 328 in the one or more sidewalls 320 of the reservoir body 318 proximate the second end 326 of the reservoir body 318. In some embodiments, the one or more openings 328 are elongated in a direction extending from the first end 324 of the reservoir body 318 to the second end 326 of the reservoir body 318 and are spaced apart from one another along a perimeter of the reservoir body 318.

In embodiments, an emulsifying buffer 330 is provided within the reservoir cavity 322 and retained therein by a buffer plunger 332 provided at the second end 326 of the reservoir body 318. In some embodiments, the buffer plunger 332 is engaged with the one of the sidewalls 320 of the reservoir body 318. In embodiments, the buffer plunger 332 includes a plunger seal 334 provided at the second end 326 of the reservoir body 318. The buffer plunger 332, in embodiments, secures the reservoir body 318 within the main body cavity 312. In embodiments, the plunger seal 334 is an elastomeric gasket or the like positioned at least partially between sidewalls 320 of the reservoir body 318 and the main body wall 314 to provide a friction fit between the reservoir body 318 and the main body wall 314. The plunger seal 334 defines an opening 336 to permit the sample collection member 304 to extend through the plunger seal 334 and enter the reservoir cavity 322. As described in more detail herein, the reservoir body 318 is permitted to move between a retracted position and a plunging position within the main body cavity 312. When moving from the retracted position to the plunging position, the reservoir body 318 moves from in a direction toward the removal end 310 of the main body 302 upon a force being applied by the sample collection member 304 against the first end 324 of the reservoir body 318.

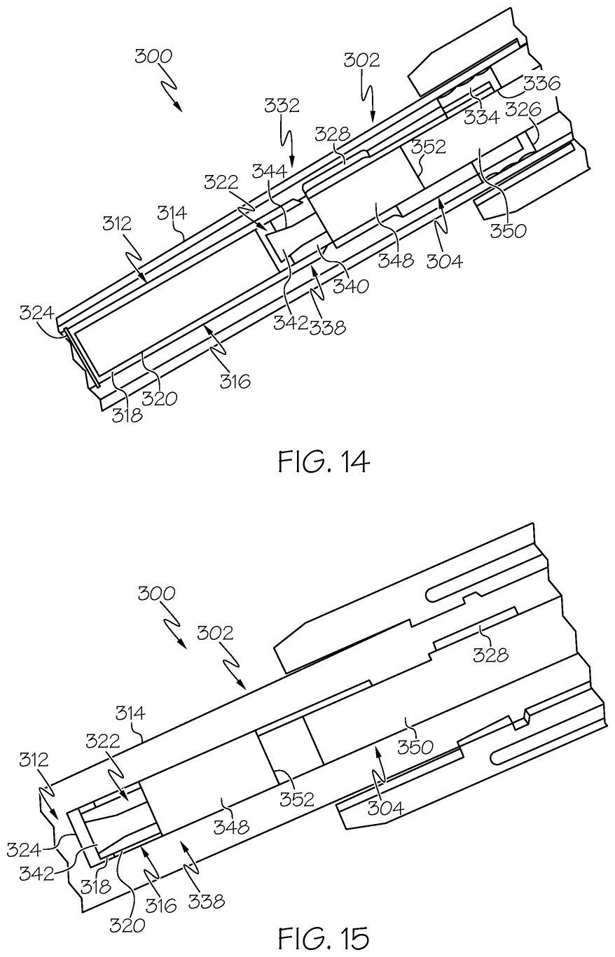

The buffer plunger 332, in embodiments, includes a buffer valve 338 for permitting the emulsifying buffer 330 to be dispensed out of the reservoir cavity 322 through the one or more openings 328 in the sidewalls 320 of the reservoir body 318 upon application of a force by the sample collection member 304 actuating the buffer valve 338. As best shown in FIGS. 13 and 14, the buffer valve 338 includes a securement portion 340 and a releasable portion 342 movable relative to the securement portion 340 and is operable positionable between a closed position and an open position. Upon application of force exceeding a first threshold by the sample collection member 304 against the releasable portion 342, the releasable portion 342 moves relative to the securement portion 340 to position the buffer valve 338 in the open position from the closed position. In the open position, a space 344 is defined between the releasable portion 342 and the securement portion 340. As described in more detail herein, the emulsifying buffer 330 is permitted to flow through the space 344 defined between the releasable portion 342 and the securement portion 340, either around or through the sample collection member 304, and out of the reservoir cavity 322 through the openings 328. Additionally, the buffer valve 338 is movable within the reservoir cavity 322 upon a force exceeding a second threshold greater than the first threshold being applied by the fecal engagement portion of the sample collection member, as described herein.

Referring again to FIG. 10, in some embodiments, a filter 346 is positioned within the main body cavity 312 between the first end 324 of the buffer solution reservoir 316 and the removal end 310 of the main body 302. The filter 346 may be formed from a porous material or have a plurality of channels formed therein to permit particles having a diameter equal to or less than a predetermined diameter mixed with the emulsifying buffer 330 to pass through the filter 346 and toward the removal end 310 of the main body 302. As such, particles having a diameter greater than the predetermined diameter defined by the filter 346 remain on the same side of the filter 346 as the buffer solution reservoir 316.

Referring still to FIG. 10, the plug 306 is releasably coupled to the removal end 310 of the main body 302 and positionable from a closed position in which the plug 306 restricts fluid from passing from the main body cavity 312 through the removal end 310, to an open position. Accordingly, the plug 306 prevents the emulsifying buffer 330 from being dispensed out of the removal end 310 of the main body 302 when coupled to the removal end 310 of the main body 302 and in the closed position. It should be appreciated that the plug 306 may be similar to the plug 106 described herein. Accordingly, in embodiments, the plug 306 may be threadedly engaged with the main body 302 in the manner discussed herein. In some embodiments, the plug 306 may be coupled to the main body 302 via a frangible seal. For example, the main body 302 and the plug 306, in embodiments, are a one-piece, monolithic structure and the plug 306 may be removed from the main body 302 by applying force in a direction away from the removal end 310 of the main body 302.

The sample collection member 304 is positionable within the main body 302 through the insertion end 308. The sample collection member 304 may be similar to the sample collection member 104 described hereinabove. With more particularity, the sample collection member 304 defines a fecal engagement portion 348 for collecting and retaining a fecal sample and an elongated member 350 extending from the fecal engagement portion 348. The elongated member 350 includes a fecal engagement end 352 and a handle end 354 opposite the fecal engagement end 352. The fecal engagement portion 348 is provided at the fecal engagement end 352 of the elongated member 350. The fecal engagement portion 348 may be similar to the fecal engagement portion 168 discussed hereinabove.

In embodiments, the sample collection member 304 may include a handle portion 356 slidably positionable along the elongated member 350. The handle portion 356 may be positionable between an extended position and a plunging position to adjust a total length of the sample collection member 304. With the handle portion 356 in the extended position, the handle portion 356 extends from the elongated member 350 in a direction opposite the fecal engagement portion 348 and a fecal sample may be collected by the fecal engagement portion 348 while maintaining an increased distance between the user and the sample itself. In embodiments, the handle portion 356 may include a cap 358 provided at an end of the handle portion 356 facing the fecal engagement portion 348 and proximate the handle end 354 of the elongated member 350 when in the extended position.

Once the elongated member 350 is inserted into the main body 302, the handle portion 356 may be unlocked, such as by rotating the handle portion 356 relative to the elongated member 350 from a locked position, to slide the handle portion 356 along the elongated member 350 and into the plunging position from the extended position, thereby reducing the total length of the sample collection member 304. Once in the plunging position, the handle portion 356 may be rotated again such that the cap 358 engages the insertion end 308 of the main body 302 to seal the main body 302. In embodiments, the cap 358 may lockingly engage the insertion end 308 of the main body 302 either upon contact or upon rotation of the handle portion 356 to lock the sample collection member 304 to the main body 302.

FIGS. 10-16 illustrate the various stages in which the fecal collection apparatus 300 is used. As such, operation of the fecal collection apparatus 300 will now be described. As shown in FIG. 10, the sample collection member 304 is initially utilized to collect a sample within the fecal engagement portion 348. As described herein, the handle portion 356 may be initially provided in the extended position to increase the total length of the sample collection member 304 to ease an operator in collecting a sample. In addition, the reservoir body 318 is initially in the retracted position and the buffer valve 338 is in the closed position such that an emulsifying buffer 330 is contained within the reservoir cavity 322. Additionally, the plug 306 is provided at the removal end 310 of the main body 302 to prevent any fluid from entering or exiting the main body 302.

Referring now to FIG. 11, the sample collection member 304 is shown partially inserted into the main body 302 through the insertion end 308. Specifically, the fecal engagement portion 348 is inserted into the main body 302 and extends through the plunger seal 334. It should be appreciated that an inner diameter of the opening 336 of the plunger seal 334 corresponds to an outer diameter of the fecal engagement portion 348 of the sample collection member 304. Accordingly, the plunger seal 334 ensures that a predetermined amount of the collected sample is permitted to enter the buffer solution reservoir 316. Specifically, the plunger seal 334 scrapes excess sample off of the fecal engagement portion 348 to permit the excess sample from entering the buffer solution reservoir 316.

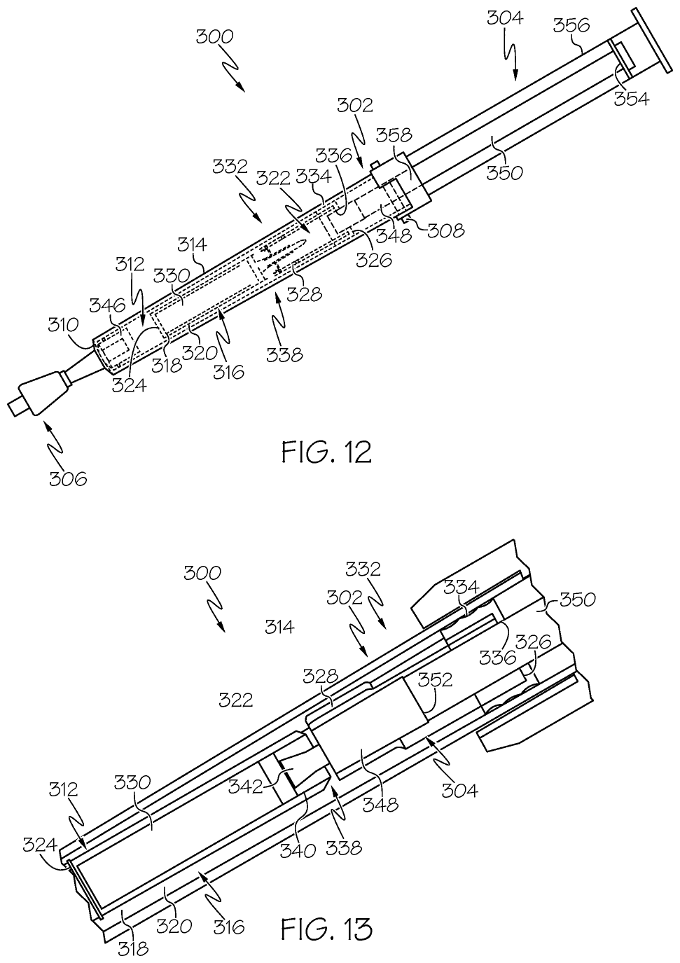

Referring now to FIG. 12, the handle portion 356 is rotated to lockingly disengage the elongated member 350 and permit the handle portion 356 to move from the extended position to the plunging position. Once the handle portion 356 is in the plunging position, the handle portion 356 may be rotated again relative to the elongated member 350 to lock the cap 358 of the handle portion 356 to the main body 302 and seal the insertion end 308 of the main body 302. It should be appreciated that the handle portion 356 may selectively disengage the cap 358 such that the sample collection member 304 may be further inserted into the main body 302 without the cap 358 inhibiting movement of the sample collection member 304.

Referring now to FIG. 13, the fecal engagement portion 348 is shown inserted further into the reservoir cavity 322 and engaging the releasable portion 342 of the buffer valve 338, which is shown in the closed position. As shown, the releasable portion 342 may be insertable into an end of the fecal engagement portion 348. With the fecal engagement portion 348 inserted further into the reservoir cavity 322, the elongated member 350 extends through the plunger seal 334 to maintain a seal at the second end 326 of the reservoir body 318.

Referring now to FIG. 14, the sample collection member 304 is shown inserted further into the main body 302 such that the fecal engagement portion 348 pushes the releasable portion 342 toward the first end 324 of the reservoir body 318 and the buffer valve 338 into the open position. As described herein, with the buffer valve 338 in the open position, a space 344 is defined between the releasable portion 342 and the securement portion 340 to permit the emulsifying buffer 330 to flow therebetween and toward the second end 326 of the reservoir body 318.

Referring now to FIG. 15, the sample collection member 304 is shown inserted further into the reservoir cavity 322 such that the sample collection member 304 is pushed further into the main body 302 toward the removal end 310. In doing so, the buffer valve 338 is moved toward the first end 324 of the reservoir body 318 displacing the emulsifying buffer 330 toward the second end 326 of the reservoir body 318. More particularly, the emulsifying buffer 330 is directed through the space 344 formed in the buffer valve 338, around the fecal engagement portion 348 and the elongated member 350 of the sample collection member 304, and out of the reservoir cavity 322 through the one or more openings 328 in the sidewalls 320 of the reservoir body 318. As the emulsifying buffer moves around the fecal engagement portion 348, the emulsifying buffer 330 mixes with the sample collected by the fecal engagement portion 348 to form a mixture of the emulsifying buffer 330 and collected sample and is stored in the main body cavity 312 between the main body wall 314 and the reservoir body 318, thus outside of the reservoir cavity 322.

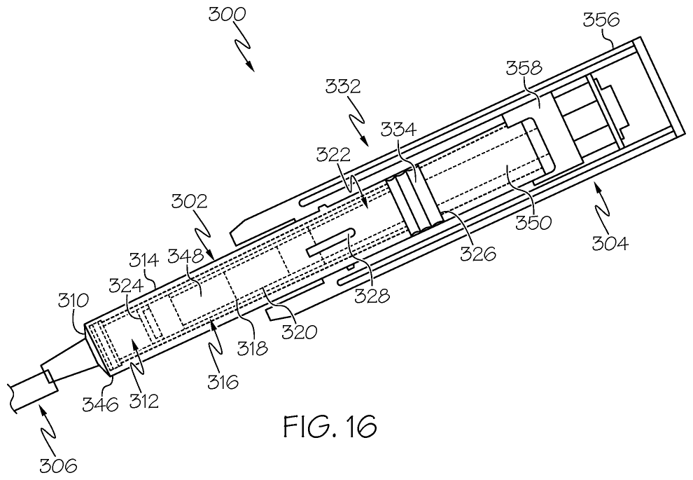

Referring now to FIG. 16, the mixture of the emulsifying buffer 330 and the collected sample is displaced out of the reservoir cavity 322 and provided between the main body wall 314 and the reservoir body 318. It should be appreciated that the plunger seal 334 maintains a fluid tight seal between the reservoir body 318 and the main body wall 314 to prevent the mixture of the emulsifying buffer 330 and the collected sample from flowing out of the main body cavity 312 and toward the insertion end 308 of the main body 302. The plunger seal 334 further creates a vacuum within the main body 302 between the main body wall 314 and the reservoir body 318.

To plunge the mixture of the emulsifying buffer 330 and the sample mixture out of the fecal collection apparatus 300, the plug 306 is removed from the removal end 310 of the main body 302 such as, for example, by rotating the plug 306 if threadedly secured or separating from the main body 302 if a frangible seal is provided. Thereafter, the sample collection member 304 is pushed further into the main body 302 toward the removal end 310 to direct the mixture through the filter 346 provided at the removal end 310. As the mixture passes through the filter 346, particulates greater than a predetermined diameter are prevented from passing through the filter 346 and the remaining mixture is emitted out of the main body 302 through the removal end 310.

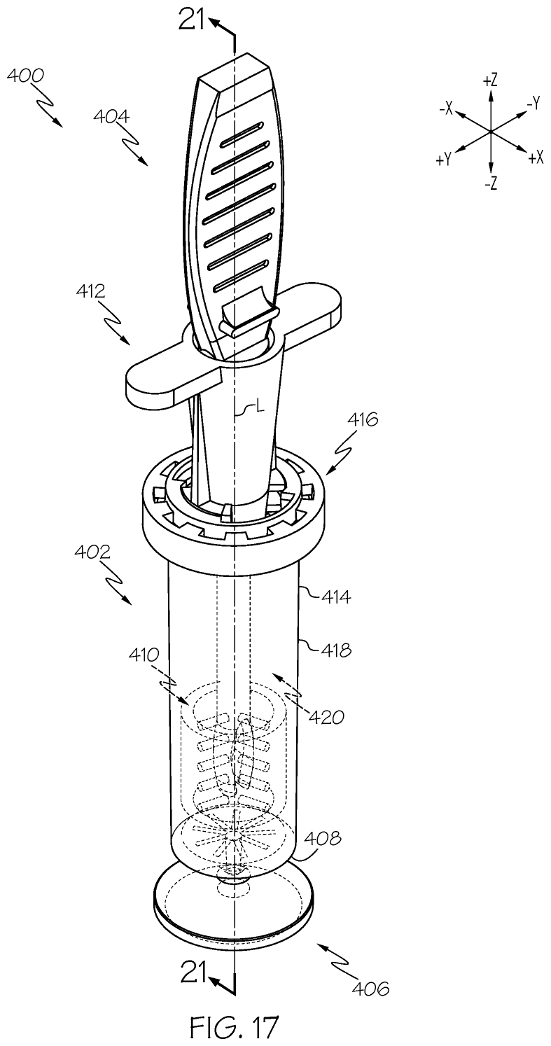

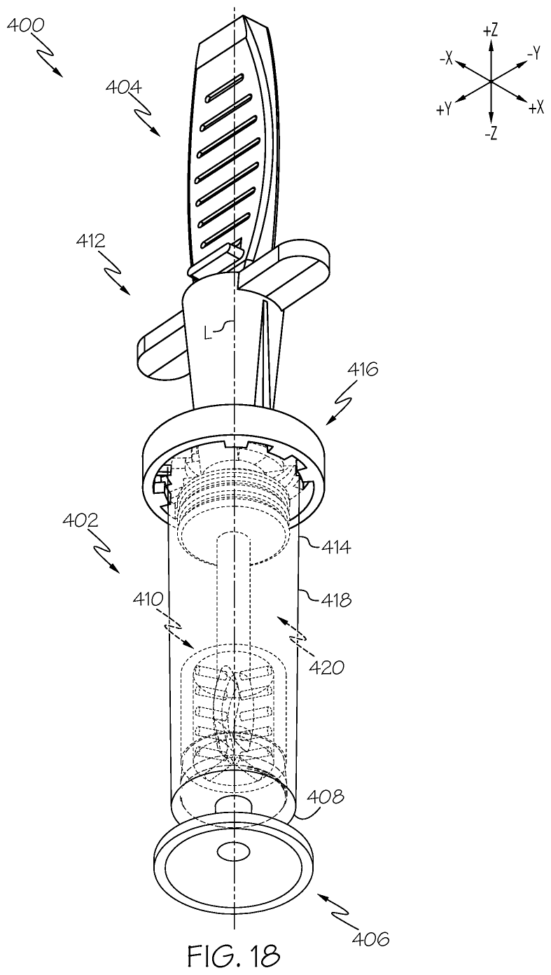

Referring now to FIGS. 17 and 18, a fecal collection apparatus 400 is illustrated according to one or more embodiments described herein. In embodiments, the fecal collection apparatus 400 generally includes a main body 402, a sample collection member 404 for collecting a fecal sample and positionable within the main body 402, and a plug 406 releasably coupled to a removal end 408 of the main body 402. It should be appreciated that an emulsifying buffer, agent, or solution may be initially provided within the main body 402, and the plug 406 prevents the emulsifying buffer from being dispensed out of the removal end 408 of the main body 402. In some embodiments, a brush 410 is provided within the main body 402 and facilitates scraping of the sample collection member 404 to remove the fecal sample from the sample collection member 404 and mix the fecal sample with the emulsifying buffer.

In embodiments, the fecal collection apparatus 400 includes a plunger 412 positioned at least partially within the main body 402 through an insertion end 414 of the main body 402 opposite the removal end 408. The sample collection member 404 is insertable through the plunger 412 and into the main body 402. In some embodiments, the fecal collection apparatus 400 includes a guide cap 416 positioned at the insertion end 414 of the main body 402. The guide cap 416, in embodiments, is engaged with the plunger 412. As described in more detail herein, in embodiments, the guide cap 416 is rotatably secured to the main body 402. In embodiments, the plunger 412 is rotatable relative to the guide cap 416 so as to permit the plunger 412 to be positionable between an unlocked state and a locked state. In the unlocked state, the plunger 412 is permitted to be inserted further into the main body 402 by application of a force. In the locked state the plunger 412 is prevented from being further inserted into the main body 402 by application of a force. With the plug 406 removed from the removal end 408 of the main body 402 and the plunger 412 positioned into the unlocked state, movement of the plunger 412 causes the emulsifying buffer mixed with the fecal sample to pass out of the main body 402 through the removal end 408. In some embodiments, the main body 402 is a syringe, and more particularly, a syringe body. Moreover, the guide cap 416, the plunger 412, and the sample collection member 404 may define a fecal collection attachment device positionable as a retrofit onto a syringe body, such as the main body 402.

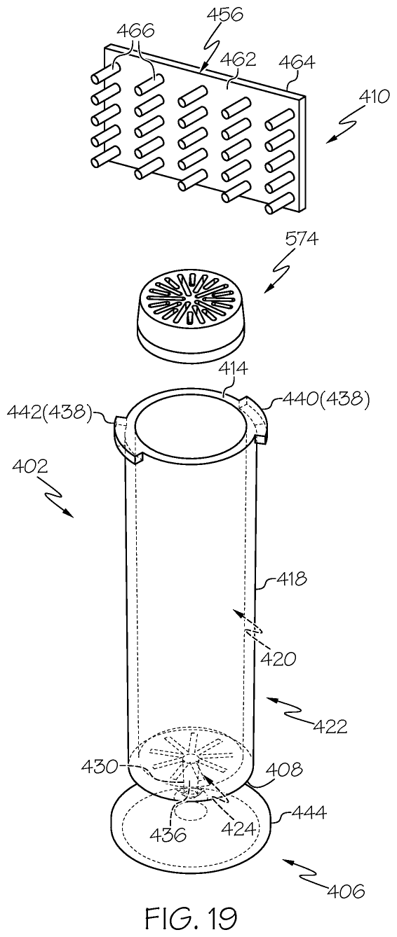

Referring now to FIG. 19, an exploded view of the main body 402, the plug 406, the brush 410, and a filter 574 are shown in greater detail. As described herein, the plug 406 is positionable at the removal end 408 of the main body 402 on top of the filter 574 to prevent emulsifying buffer from flowing out of the removal end 408 of the main body 402. The brush 410 (shown in an expanded or unfolded position in FIG. 19) is positionable within the main body 402.

With respect to the main body 402, the main body 402 includes a main body wall 418 defining a main body cavity 420. The main body wall 418 includes the removal end 408 provided at a lowermost point of the main body 402 and the insertion end 414 provided at an uppermost point of the main body 402 opposite the removal end 408. The main body 402 further defines a longitudinal axis L extending in the +/−Z direction of the coordinate axes depicted in the drawings.

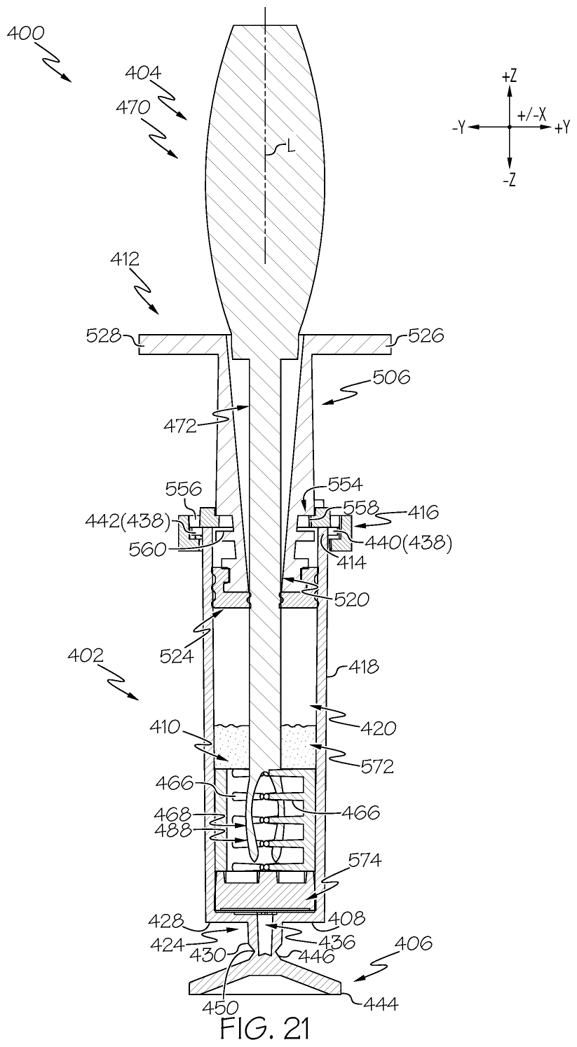

In embodiments, as shown in FIGS. 19 and 21, the main body 402 includes an upper body portion 422, which includes the insertion end 414, and a lower body portion 424, which includes the removal end 408. In embodiments (not depicted), an outer diameter of the lower body portion 424 is equal to or greater than an outer diameter of the upper body portion 422. In other embodiments, as shown, the outer diameter of the lower body portion 424 is less than the outer diameter of the upper body portion 422. In embodiments, the upper body portion 422 includes a base wall 428 and the lower body portion 424 is defined by a longitudinal wall 430 extending from the base wall 428. The longitudinal wall 430 of the lower body portion 424 defines a conduit 436 extending through the base wall 428 to place the lower body portion 424 in fluid communication with the main body cavity 420 of the upper body portion 422. Thus, the emulsifying buffer is permitted to flow out of the removal end 408 of the main body 402 from the main body cavity 420 of the upper body portion 422 through the conduit 436 defined by the longitudinal wall 430 of the lower body portion 424.

In some embodiments, the main body 402 includes a flange 438 provided at the insertion end 414 thereof and extending radially relative to the longitudinal axis L of the main body 402. That is, the flange 438 extends in a direction generally perpendicular to the longitudinal axis L of the main body 402. In embodiments, the flange 438 is provided at the insertion end 414 of the main body 402 and includes a first flange portion 440 extending in a first radial direction relative to the longitudinal axis L from the insertion end 414 of the main body 402 and a second flange portion 442 extending in a second radial direction from the insertion end 414 of the main body 402. In embodiments, the first flange portion 440 and the second flange portion 442 are diametrically opposed to one another and extend in opposite radial directions, however this is merely an example. Although the flange 438 depicted herein includes a pair of flange portions 440, 442, it should be appreciated that any number of flange portions may be provided. For example, the flange 438 may include a single flange portion or more than two flange portions. In embodiments, in which more than two flange portions are provided, the flange portions may be spaced apart from one another in any suitable arrangement such as, for example, equidistantly from one another in a circumferential direction.

Referring still to FIGS. 19 and 21, the plug 406 is shown attached to the main body 402. In the embodiment depicted in FIGS. 19 and 21, the plug 406 includes a gripping portion 444 and an extension portion 446 extending from the gripping portion 444.

In embodiments, the plug 406 may be integrally formed as a one-piece, monolithic structure with the main body 402 at the removal end 408 of the main body 402. Accordingly, a frangible seal 450 may be formed in either the plug 406 or the main body 402 where the plug 406 and the main body 402 meet. The frangible seal 450 may include, for example, a perforated edge, plurality of cuts, or the like, to permit the plug 406 to be easily removed from the main body 402 such as by applying force in a direction away from the removal end 408 of the main body 402.

In other embodiments, the plug 406 may be press-fitted within the removal end 408 of the main body 402 or threadedly engaged with the removable end 408 of the main body 402, as described in other embodiment depicted herein. Regardless of the specific physical implementation of the connection, it should be understood that the plug 406 is removable from the lower body portion 424 of the main body 402 by applying a force against the plug 406.

Referring still to FIG. 19, the brush 410 is illustrated separate from the main body 402. The brush 410 is positionable between an unfolded position when not positioned within the main body 402, as shown in FIG. 19, and a folded position when positioned within the main body 402, as shown in FIGS. 17 and 18. The brush 410 includes a brush body 456 having a first surface 462 and a second surface 464 opposite the first surface 462. The brush body 456 also includes one or more bristles 466 extending from the first surface 462 in a direction perpendicular from a plane of the main body 402. Accordingly, when the brush 410 is in the folded position, the bristles 466 each extends in a radial direction toward one another. The bristles 466 may be provided in any suitable arrangement along the first surface 462 of the brush body 456. For example, the bristles 466 may be arranged in one or more rows. In embodiments, the brush body 456 and the bristles 466 may each be formed from a flexible plastic, rubber, or the like, so as to allow the brush body 456 to fold into the folded position.

The filter 574 may be positioned within the main body cavity 420 between the brush 410 and the base wall 428 of the lower body portion 424. The filter 574 may be formed from a porous material or have a plurality of channels formed therein to permit particles having a diameter equal to or less than a predetermined diameter mixed with the emulsifying buffer to pass through the filter 574 and toward the removal end 408 of the main body 402. As such, in use, particles having a diameter greater than the predetermined diameter defined by the filter 574 remain on the same side of the filter 574 as the brush 410.

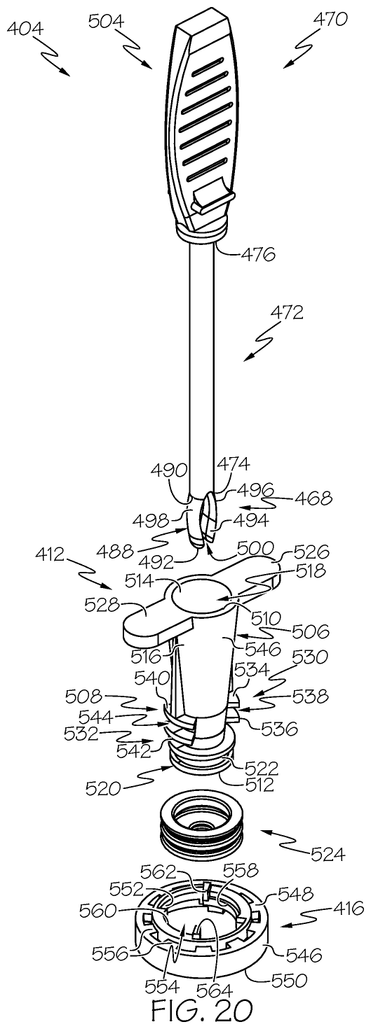

Referring now to FIG. 20, an exploded view of the sample collection member 404, the plunger 412, and the guide cap 416 are shown in greater detail. As described in detail herein, the sample collection member 404, the plunger 412, and the guide cap 416 are positioned at the insertion end 414 of the main body 402.

With respect to the sample collection member 404, the sample collection member 404 defines a fecal engagement portion 468 for collecting and retaining a fecal sample and a handle portion 470 spaced apart from the fecal engagement portion 468. In embodiments, the sample collection member 404 includes an elongated member 472 having a fecal engagement end 474 and a handle end 476 opposite the fecal engagement end 474. The fecal engagement portion 468 is provided at the fecal engagement end 474 of the elongated member 472 and the handle portion 470 is provided at the handle end 476 of the elongated member 472. In embodiments, the fecal engagement portion 468, the handle portion 470, and the elongated member 472 are formed as a one-piece, monolithic structure. In embodiments, as described in other embodiments herein, the elongated member 472 has a tapered portion located at a substantially central location of the elongated member 472. In some embodiments, as also described in other embodiments herein, a groove is formed in the elongated member 472 proximate the handle end 476 of the elongated member 472 and a sealing member, such as an O-ring, is provided within the groove for providing a fluid tight seal with the plunger 412.

The fecal engagement portion 468 is deformable between an uncompressed position and a compressed position. The fecal engagement portion 468 includes one or more pincers 488 extending from the fecal engagement end 474 of the elongated member 472. As shown in FIG. 20, the fecal engagement portion 468 includes two pincers 488 spaced apart from one another. However, it should be appreciated that the fecal engagement portion 468 may include any suitable number of pincers 488 for collecting a fecal sample therebetween. For example, the fecal engagement portion 468 may include one pincer 488 for collecting a fecal sample. In other embodiments, the fecal engagement portion 468 may include three or more pincers 488. Each pincer 488 includes a proximal end 490 extending from the elongated member 472, a distal end 492 opposite the proximal end 490, an inner surface 494, and an outer surface 496 opposite the inner surface 494. In embodiments, the pincers 480 may be spaced apart from one another to define a receptacle 500 in which the fecal sample is retained. In embodiments, each pincer 488 may have an arcuate shape such that a distance between opposite pincers 488 decreases further along a length of the pincers 488 in a direction opposite elongated member 472 (i.e., in the −Z-direction as depicted). In this embodiment, each pincer 488 includes a widened point 498 formed at a substantial medial location between the proximal end 490 and the distal end 492 of the pincer 488. As such, the receptacle 500 defined by the pincers 488 may have a substantially elliptical geometry extending from the proximal end 490 and the distal end 492 of the pincers 488. The fecal engagement portion 468 may be formed from a deformable or flexible material, such as a flexible plastic, rubber, or the like, to permit the pincers 488 to compress toward one another upon application of a force in a direction toward the receptacle 500 from the outer surface 496 of the pincers 488. In other embodiments, the fecal engagement portion 468 may be formed from a substantially rigid material, but flexibly joined to the elongated member 472 to allow the pincers 488, and more particularly the distal end 492 of the pincers 488, to compress toward one another upon application of a force.

With the fecal engagement portion 468 in the uncompressed position, the fecal engagement portion 468 defines an uncompressed sample collection outer diameter between the outer surface 496 of each pincer 488 and at the widened point 498 of each pincer 488. With the fecal engagement portion 468 in the compressed position, such as when the fecal engagement portion 468 is being inserted through the plunger 412, the fecal engagement portion 468 defines a compressed sample collection outer diameter between the outer surface 496 of each pincer 488 and at the widened point 498 of each pincer 488 that is less than the uncompressed sample collection outer diameter when the fecal engagement portion 468 is in the uncompressed position.

The handle portion 470 is provided at the handle end 476 of the elongated member 472 opposite the fecal engagement portion 468. The handle portion 470 includes a tab 504 extending in a direction opposite the elongated member 472. In embodiments, as shown, the tab 504 has a concave surface conforming to a shape of a thumb or finger of a user. However, it should be appreciated that the tab 504 may have any suitable shape.

Referring still to FIG. 20, the plunger 412 includes a plunger body 506 and a plunger guide 508. The plunger body 506 has an upper end 510, a lower end 512 opposite the upper end 510, an inner surface 514, and an outer surface 516 opposite the inner surface 514 extending between the upper end 510 and the lower end 512. In embodiments, the inner surface 514 and the outer surface 516 are each tapered from the upper end 510 toward the lower end 512. The inner surface 514 of the plunger body 506 defines an open-ended channel 518 and an end of the channel 518 opposite the lower end 512 of the plunger body 506 defines a sample collection aperture 520. The sample collection aperture 520 defines an aperture inner diameter. In embodiments, a groove 522 is defined in the outer surface 516 of the plunger body 506 proximate the lower end 512 of the plunger body 506 and a sealing member 524, such as an O-ring, gasket, or the like, is provided within the groove 522 to provide a fluid tight seal between the outer surface 516 of the plunger body 506 and the main body 402.

In embodiments, a first flange 526 and a second flange 528 are provided at the upper end 510 of the plunger body 506 and extends radially therefore. As shown, the first flange 526 extends in a first radial direction and the second flange 528 extends in a second radial direction opposite the first radial direction. As such, in embodiments, the first flange 526 and the second flange 528 are diametrically opposed to one another and extend in opposite radial directions, however this is merely an example. Although a pair of flanges 526, 528 are depicted herein, it should be appreciated that any number of flanges may be provided. For example, the plunger body 506 may include a single flange or more than two flanges. In embodiments, in which more than two flanges are provided, the flanges may be spaced apart from one another in any suitable arrangement such as, for example, equidistantly from one another in a circumferential direction.

Referring still to FIG. 20, the plunger guide 508 is formed on the outer surface 516 of the plunger body 506 proximate the lower end 512 of the plunger body 506, i.e., closer to the lower end 512 and the upper end 510, and spaced apart from the grove 522. However, it should be appreciated that the plunger guide 508 may be located at any location along the plunger body 506. The plunger guide 508 includes at least one slotted portion. As shown, the plunger guide 508 includes a first slotted portion 530 provided on one side of the outer surface 516 of the plunger body 506, and a second slotted portion 532 formed on an opposite side of the outer surface 516 of the plunger body 506. The first slotted portion 530 includes a first upper wall 534 and a first lower wall 536 defining a first slot 538. Similarly, the second slotted portion 532 includes a second upper wall 540 and a second lower wall 542 defining a second slot 544. Although the plunger guide 508 is illustrated as including the first slotted portion 530 and the second slotted portion 532, it should be appreciated that the plunger guide 508 may include any number of slotted portions, such as one slotted portion or more than two slotted portions.

Referring still to FIG. 20, the guide cap 416 includes an outer wall 546 having an upper end 548, a lower end 550, and an inner surface 552. An opening 554 is defined by the outer wall 546 and a plurality of teeth 556 extend radially inwardly from the inner surface 552 of the outer wall 546 into the opening 554. As described in more detail herein, the opening 554 permits the guide cap 416 to be positioned on the main body 402 (FIG. 19) at the insertion end 414 and permits the flange 438 (FIG. 19) of the main body 402 to extend into the opening 554 of the guide cap 416 to engage the plurality of teeth 556 of the guide cap 416 and retain the guide cap 416 on the insertion end 414 of the main body 402. The guide cap 416 further includes a first rail 558 and a second rail 560 formed on the inner surface 552 of the outer wall 546 of the guide cap 416. The first rail 558 and the second rail 560 are formed on opposite sides of the inner surface 552 of the outer wall 546 of the guide cap 416. As described in more detail herein, the first rail 558 and the second rail 560 correspond to the first slotted portion 530 and the second slotted portion 532, respectively. Accordingly, the number and position of the first rail 558 and the second rail 560 correspond to the number and position of the first slotted portion 530 and the second slotted portion 532. In embodiments, a first stopper 562 is provided at an end of the first rail 558, and a second stopper 564 is provided at an end of the second rail 560. The first stopper 562 has a thickness extending in an axial direction and/or a width extending in a radial direction greater than a corresponding thickness and width of the first rail 558. Similarly, the second stopper 564 has a thickness extending in an axial direction and/or a width extending in a radial direction greater than a corresponding thickness and width of the second rail 560. Although it is described herein that the first slotted portion 530 and the second slotted portion 530 are provided on the plunger guide 508, and the first rail 558 and the second rail 560 are provided on the guide cap 416, it should be appreciated that the opposite may be true. For example, the first slotted portion 530 and the second slotted portion 532 may be formed on the guide cap 416, and the first rail 558 and the second rail 560 may be formed on the plunger guide 508.

Referring now to FIG. 21, a cross-section view of the fecal collection apparatus 400 is shown in an assembled state. As shown, in the assembled state, the plug 406 positioned at the removal end 408 of the main body 402. As described herein, the plug 406 may be press fitted into the removal end 408 of the main body 402, integrally formed therewith as a one piece, monolithic structure and detachable from the main body 402 at the frangible seal 450, or threadedly engaged with the main body 402. The plug 406 can be removed from the main body 402 upon application of force applied at the frangible seal 450. As shown, emulsifying buffer 572 is provided into the main body cavity 420 and retained therein by the plug 406, which prevents the emulsifying buffer 572 from exiting the main body 402 through the conduit 436 of the lower body portion 424.

As shown in FIG. 21, the brush 410 is positioned within the main body cavity 420. In the embodiment shown in FIG. 21, the brush 410 is positioned proximate the removal end 408 of the main body 402. The brush 410 is shown in the folded position within the main body cavity 420 such that the bristles 466 are directed inwardly.

In use, the guide cap 416 is positioned onto the insertion end 414 of the main body 402 by inserting the insertion end 414, particularly the flange 438 of the main body 402, through the opening 554 of the guide cap 416. The guide cap 416 may be secured by rotating or press-fitting the guide cap 416 relative to the main body 402 such that the flange 438 is retained within the teeth 556 of the guide cap 416. Once secured, the guide cap 416 is prohibited from rotating or moving along the longitudinal axis L of the main body 402.

Referring still to FIG. 21, the plunger 412 is shown inserted into the insertion end 414 of the main body 402. With the plunger body 506 positioned into the main body cavity 420, the sealing member 524 circumscribing the outer surface 496 of the plunger body 506 proximate the lower end 512 thereof provides a fluid tight seal between the outer surface 496 of the plunger body 506 and the main body wall 418 of the main body 402. As described herein, when the plunger 412 is in the locked state, the plunger 412 is restricted from moving along the longitudinal axis L of the main body 402 in a direction toward the insertion end 414 of the main body 402.

Thereafter, the sample collection member 404 may be utilized to collect a fecal sample, such as by inserting the fecal engagement portion 468 into a fecal matter, and collecting the fecal sample within the receptacle 500 between the pincers 488. The sample collection member 404 may then be inserted into the main body 402 through the sample collection aperture 520 formed in the plunger body 506 and through the insertion end 414 of the main body 402 into the main body cavity 420. Prior to inserting the sample collection member 404 into the plunger 412, the fecal engagement portion 468 is in the uncompressed position with the pincers 488 spaced apart from one another to define the uncompressed sample collection outer diameter. As described herein, the uncompressed sample collection outer diameter of the plunger body 506 is greater than the aperture inner diameter of the sample collection aperture 520. Therefore, as the fecal engagement portion 468 is inserted into the plunger 412 and contacts the inner surface 514 of the plunger body 506 at the sample collection aperture 520, the inner surface 514 of the plunger body 506 compresses the pincers 488 of the fecal engagement portion 468 to position the fecal engagement portion 468 into the compressed position. As described herein, when the fecal engagement portion 468 is in the compressed position, the fecal engagement portion 468 has a compressed sample collection outer diameter that is less than the aperture inner diameter. Thus, the fecal engagement portion 468 is permitted to pass through the sample collection aperture 520. In doing so, excess fecal sample clinging to an outside of the fecal engagement portion 468 is retained within the plunger 412, rather than being further inserted into the main body cavity 420 and mixing with the emulsifying buffer 572. This ensures that only a controlled amount of fecal matter is permitted to mix with the emulsifying buffer 572 within the main body cavity 420. Once the sample collection member 404 is inserted into the main body 402, the elongated member 472 of the sample collection member 404 forms a fluid tight seal with the inner surface 514 of the plunger body 506. Thus, the emulsifying buffer 572 is prevented from flowing through the channel 518 formed in the plunger 412 and out of the insertion end 414 of the main body 402.

To mix the fecal sample with the emulsifying buffer 572, the sample collection member 404 is moved in opposite directions along the longitudinal axis L of the main body 402 throughout the main body cavity 420 and rotated. In doing so, the bristles 466 of the brush 410 scrape the fecal sample out of the fecal engagement portion 468 of the sample collection member 404 so that the fecal sample mixes with the emulsifying buffer 572.

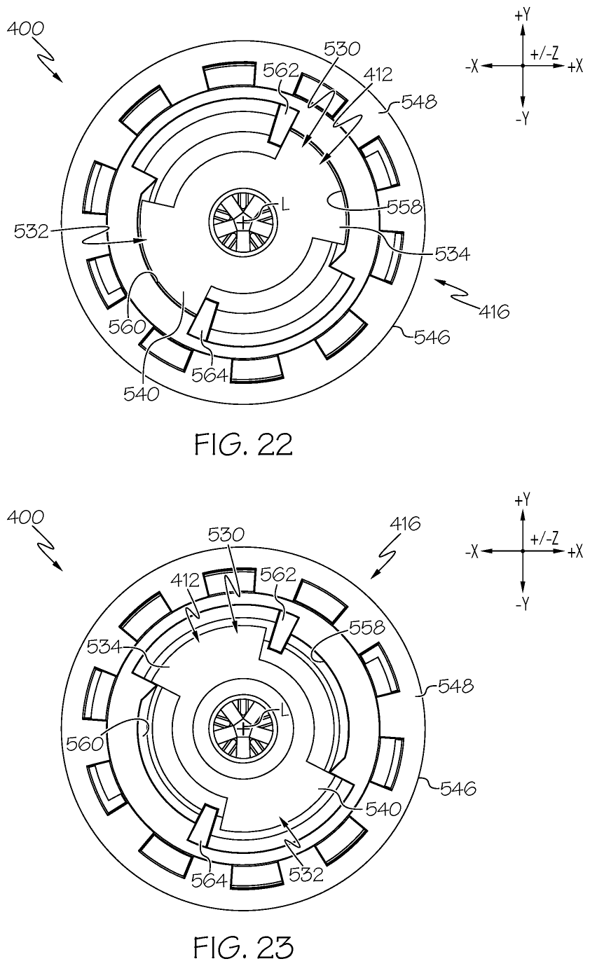

Referring now to FIGS. 22 and 23, the plunger 412 is shown in the locked state and the unlocked state, respectively. When in the locked state, as shown in FIG. 22, the first rail 558 of the plunger 412 is received within the first slot 538 of the first slotted portion 530, and the second rail 560 is received within the second slot 544 of the second slotted portion 532. More specifically, when the plunger 412 is in the locked state, the first rail 558 is positioned between the first upper wall 534 and the first lower wall 536 of the first slotted portion 530, and the second rail 560 is positioned between the second upper wall 540 and the second lower wall 542 of the second slotted portion 532. In the locked state, the first upper wall 534 of the first slotted portion 530 abuts against the first stopper 562, and the second upper wall 540 of the second slotted portion 532 abuts against the second stopper 564 to permit rotation of the plunger 412 in only one direction.

In the unlocked state, as shown in FIG. 23, the plunger 412 is rotated in a direction away from the first stopper 562 and the second stopper 564 such that the first rail 558 is moved out of the first slot 538 and the second rail 560 is moved out of the second slot 544. With the plunger 412 in the unlocked state, the sealing member 524 and/or the second end 512 of the plunger 412 contacts the brush 410 or, alternatively, the first flange 526 and the second flange 528 contact the upper end 548 of the guide cap 416.

As described herein with respect to other embodiments, indicia may be provided on the outer wall 546 of the guide cap 416 to provide a visual indication of a position or state of the plunger 412. The indicia may include a lock symbol to indicate that the plunger 412 is in the locked state, and an unlock symbol to indicate that the plunger 412 is in the unlocked state.

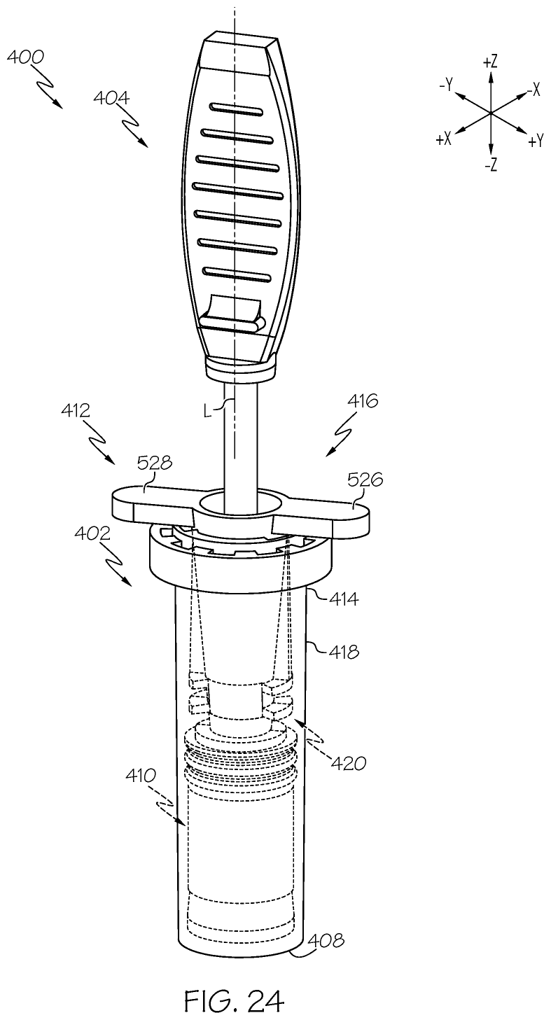

To dispense the mixture of emulsifying buffer 572 and fecal sample out of the removal end 408 of the main body 402, the plunger 412 is rotated into the unlocked state and pressed toward the removal end 408 of the main body 402 along the longitudinal axis L. Accordingly, as shown in FIG. 24, the plunger 412 is shown in a lowered position. In use, the sample collection member 404 may be additionally moved, either by twisting or moving in an out of the main body cavity 420 independently of the plunger 412 to facilitate the brush 410 removing fecal sample from the sample collection member 404 and mixing with the emulsifying buffer 572. Additionally, the plunger 412 may alternate between the raised position and the lowered position to dispense an additional supply of mixture of the emulsifying buffer 572 and the fecal sample. In embodiments, the sample collection member 404 may be withdrawn from the main body cavity 420 prior to moving the plunger 412 into the lowered position and a cap may be inserted into the plunger 412 to prevent the emulsifying buffer 572 from escaping from the main body 402.

From the above, it is to be appreciated that defined herein is a fecal collection apparatus that a main body, a sample collection member positionable within the main body, and a plug provided at a removal end of the main body to restrict fluid from passing through the main body through from removal end. The sample collection member includes a fecal engagement portion that facilitates the collection of a predetermined amount of fecal matter to be mixed with an emulsifying buffer within the main body. As described herein, the fecal collection apparatus may include a plunger to control a predetermined amount of dispensing of an emulsifying buffer out of the main body that has mixed with the collected fecal sample. In addition, a fecal collection attachment device is disclosed that may be coupled to an end of a main body or syringe as a retrofit attachment.

Further aspects of the embodiments described herein are provided by the subject matter of the following clauses:

Clause 1. A fecal collection apparatus comprising: a main body including an insertion end and a removal end opposite the insertion end, the main body defining a main body cavity between the insertion end and the removal end; a sample collection member positionable within the main body cavity through the insertion end, the sample collection member defining a fecal engagement portion for holding a fecal sample and a handle portion spaced apart from the fecal engagement portion; and a plug releasably coupled to the removal end of the main body, the plug positionable from a closed position in which the plug restricts fluid from pas sing from the main body cavity through the removal end, to an open position.

Clause 2. The fecal collection apparatus of clause 1, further comprising a plunger positioned at least partially within the main body cavity of the main body.

Clause 3. The fecal collection apparatus of clause 2, wherein the plunger defines a sample collection aperture defining an aperture inner diameter, the sample collection member insertable through the sample collection aperture.

Clause 4. The fecal collection apparatus of clause 3, wherein the fecal engagement portion is deformable between a compressed position and an uncompressed position, the fecal engagement portion defining an uncompressed sample collection outer diameter when in the uncompressed position that is greater than the aperture inner diameter, and a compressed sample collection outer diameter when in the compressed position that is less than the uncompressed sample collection outer diameter.

Clause 5. The fecal collection apparatus of any one of clauses 2-4, further comprising a guide cap positioned at the insertion end of the main body.

Clause 6. The fecal collection apparatus of clause 5, wherein: the guide cap comprises a lower retaining groove; the main body includes a flange extending from the insertion end; and the flange of the main body is insertable into the lower retaining groove to retain the guide cap on the main body.

Clause 7. The fecal collection apparatus of clause 5 or clause 6, wherein the guide cap defines an upper retaining groove defining a first groove portion having a first height extending in an axial direction corresponding to a longitudinal axis extending from the insertion end to the removal end of the main body, and a second groove portion having a second height extending in the axial direction, the second height being greater than the first height.

Clause 8. The fecal collection apparatus of clause 7, wherein: the plunger comprises a finger extending in a radial direction and partially insertable into the upper retaining groove; and the plunger is rotatable relative to the guide cap to move the finger between the first groove portion and the second groove portion.

Clause 9. The fecal collection apparatus of clause 8, wherein the sample collection member and the plunger are inhibited from moving in the axial direction when the finger is within the first groove portion, and wherein the sample collection member and the plunger are permitted to move in the axial direction when the finger is within the second groove portion.

Clause 10. The fecal collection apparatus of any one of clauses 1-9, further comprising a brush positioned within the main body cavity of the main body proximate the removal end, the brush having a plurality of bristles for engaging the fecal engagement portion of the sample collection member.

Clause 11. The fecal collection apparatus of any one of clauses 1-10, further comprising an emulsifying buffer positioned in the main body cavity.

Clause 12. The fecal collection apparatus of any one of clauses 1-11, wherein the plug is coupled to the main body through a frangible seal.

Clause 13. The fecal collection apparatus of any one of clauses 1-12, wherein the plug is monolithic with the main body.

Clause 14. A fecal collection apparatus comprising: a main body comprising an insertion end and a removal end; a plunger positioned at least partially within the main body; a sample collection member positionable within the main body through the insertion end, the sample collection member defining a fecal engagement portion for holding a fecal sample; and a guide cap positioned at the insertion end of the main body, wherein: the plunger is movable with respect to the guide cap between a locked state and an unlocked state; the plunger and sample collection member are prevented from moving in an axial direction with the plunger in the locked state, the axial direction corresponding to a longitudinal axis extending from the insertion end to the removal end of the main body; and the plunger and sample collection are moveable in the axial direction with the plunger in the unlocked state.

Clause 15. The fecal collection apparatus of clause 14, wherein the plunger defines a sample collection aperture defining an aperture inner diameter.

Clause 16. The fecal collection apparatus of clause 15, wherein the sample collection member comprises a handle portion and the fecal engagement portion opposite the handle portion, the fecal engagement portion being deformable between a compressed position and an uncompressed position, the fecal engagement portion defining an uncompressed sample collection outer diameter that is greater than the aperture inner diameter when in the uncompressed position.

Clause 17. The fecal collection apparatus of any one of clauses 14-16, wherein: the guide cap comprises a lower retaining groove; and the main body includes a flange extending from the insertion end, wherein the flange of the main body is insertable into the lower retaining groove to retain the guide cap on the main body.

Clause 18. The fecal collection apparatus of any one of clauses 14-17, wherein the guide cap defines an upper retaining groove defining a first groove portion having a first height extending in an axial direction corresponding to a longitudinal axis extending from the insertion end to the removal end of the main body, and a second groove portion having a second height extending in the axial direction, the second height being greater than the first height.

Clause 19. The fecal collection apparatus of claim 18, wherein: the plunger comprises a finger extending in a radial direction and partially insertable into the upper retaining groove; and the plunger is rotatable relative to the guide cap to move the finger into the first groove portion to be in the locked state and into the second groove portion to be in the unlocked state.

Clause 20. The fecal collection apparatus of any one of clauses 14-19, further comprising a brush positioned within the main body, the brush having a plurality of bristles for engaging the fecal engagement portion of the sample collection member.

Clause 21. A fecal collection attachment device positionable onto a syringe, the fecal collection attachment device comprising: a guide cap positionable onto the syringe, the guide cap comprising an upper retaining groove defining a first groove portion and a second groove portion; a plunger comprising a finger partially insertable into the upper retaining groove, the plunger rotatable relative to the guide cap to position the finger between the first groove portion and the second groove portion; and a sample collection member positionable through the plunger, the sample collection member defining a fecal engagement portion for holding a fecal sample, wherein the plunger and the sample collection member are inhibited from moving in an axial direction relative to the guide cap when the finger is positioned within the first groove portion, and wherein the sample collection member and the plunger are permitted to move in the axial direction when the finger is positioned within the second groove portion.

Clause 22. The fecal collection attachment device of clause 21, wherein the plunger defines a sample collection aperture defining an aperture inner diameter.

Clause 23. The fecal collection attachment device of clause 22, wherein the sample collection member comprises a handle portion and the fecal engagement portion opposite the handle portion, the fecal engagement portion being deformable between a compressed position and an uncompressed position, the collection end defining an uncompressed sample collection outer diameter that is greater than the aperture inner diameter when in the uncompressed position.

Clause 24. A fecal collection apparatus comprising: a main body including an insertion end and a removal end opposite the insertion end, the main body defining a main body cavity between the insertion end and the removal end; a sample collection member positionable within the main body cavity through the insertion end, the sample collection member defining a fecal engagement portion for holding a fecal sample and a handle portion spaced apart from the fecal engagement portion; and a plug releasably coupled to the removal end of the main body, the plug positionable from a closed position in which the plug restricts fluid from passing from the main body cavity through the removal end, to an open position.

Clause 25. The fecal collection apparatus of clause 24, further comprising a buffer solution reservoir positioned within the main body cavity, the buffer solution reservoir defining one or more sidewalls.

Clause 26. The fecal collection apparatus of clause 25, further comprising a buffer plunger engaged with the one or more sidewalls of the buffer solution reservoir.

Clause 27. The fecal collection apparatus of clause 26, wherein the buffer plunger comprises a buffer valve, the buffer valve positionable in a closed position, in which the buffer valve restricts the flow of fluid through the buffer valve, and an open position, in which the buffer valve permits the flow of fluid through the buffer valve.

Clause 28. The fecal collection apparatus of clause 27, wherein the buffer valve comprises a securement portion and a releasable portion movable relative to the securement portion upon a force applied by the fecal engagement portion of the sample collection member.

Clause 29. The fecal collection apparatus of clause 27 or clause 28, wherein the buffer valve is movable within the buffer solution reservoir in a direction toward the removal end of the main body upon a force applied by the fecal engagement portion of the sample collection member.

Clause 30. The fecal collection apparatus of any one of clauses 25-29, wherein the one or more sidewalls of the buffer solution reservoir define one or more openings.

Clause 31. The fecal collection apparatus of any one of clauses 24-30, further comprising a filter positioned at the removal end of the main body.

Clause 32. The fecal collection apparatus of any one of clauses 25-31, further comprising a plunger seal positioned within the main body cavity of the main body at an end of the buffer solution reservoir.

Clause 33. The fecal collection apparatus of clause 32, wherein the plunger defines a sample collection aperture defining an aperture inner diameter, the sample collection member insertable through the sample collection aperture.

Clause 34. The fecal collection apparatus of clause 33, wherein the fecal engagement portion is deformable between a compressed position and an uncompressed position, the fecal engagement portion defining an uncompressed sample collection outer diameter when in the uncompressed position that is greater than the aperture inner diameter, and a compressed sample collection outer diameter when in the compressed position that is less than the uncompressed sample collection outer diameter.

Clause 35. The fecal collection apparatus of any one of clauses 24-34, wherein the plug is coupled to the main body through a frangible seal.

Clause 36. The fecal collection apparatus of any one of clauses 24-35, wherein the plug is monolithic with the main body.

Clause 37. A fecal collection apparatus comprising: a main body including an insertion end and a removal end opposite the insertion end, the main body defining a main body cavity between the insertion end and the removal end; a buffer solution reservoir positioned within the main body cavity, the buffer solution reservoir defining a reservoir body; a sample collection member positionable within the main body cavity and the buffer solution reservoir through the insertion end, the sample collection member defining a fecal engagement portion for holding a fecal sample; and a plug releasably coupled to the removal end of the main body, wherein the buffer solution reservoir is movable in a direction toward the removal end of the main body upon application of a force by the sample collection member.

Clause 38. The fecal collection apparatus of clause 37, further comprising a buffer plunger engaged with the buffer solution reservoir.

Clause 39. The fecal collection apparatus of clause 38, wherein the buffer plunger comprises a buffer valve, the buffer valve positionable in a closed position, in which the buffer valve restricts flow of a fluid through the buffer valve, and an open position, in which the buffer valve permits flow of the fluid through the buffer valve.

Clause 40. The fecal collection apparatus of clause 39, wherein the buffer valve comprises a securement portion and a releasable portion movable relative to the securement portion.

Clause 41. The fecal collection apparatus of clause 39 or clause 40, wherein the buffer valve is movable within the buffer solution reservoir in a direction toward the removal end of the main body.

Clause 42. The fecal collection apparatus of any one of clauses 37-41, wherein a plurality of openings are formed in one or more sidewalls of the buffer solution reservoir.

Clause 43. The fecal collection apparatus of any one of clauses 37-42, further comprising a filter positioned at the removal end of the main body.

Clause 44. The fecal collection apparatus of any one of clauses 37-43, further comprising a fluid stored within the buffer solution reservoir.

Clause 45. The fecal collection apparatus of clause 44, wherein the fluid is an emulsifying buffer.

Clause 46. The fecal collection apparatus of any one of clauses 37-45, further comprising a fluid stored within the buffer solution reservoir and permitted to mix with the fecal sample when the buffer valve is in the open position.

It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

What is claimed is:

1. A fecal collection apparatus comprising:
a main body including an insertion end and a removal end opposite the insertion end, the main body defining a main body cavity between the insertion end and the removal end;
a sample collection member positionable within the main body cavity through the insertion end, the sample collection member defining a fecal engagement portion for holding a fecal sample and a handle portion spaced apart from the fecal engagement portion; and
a plug provided at the removal end of the main body, the plug positionable from a closed position in which the plug restricts fluid from passing from the main body cavity through the removal end, to an open position.

2. The fecal collection apparatus of claim 1, further comprising a plunger positioned at least partially within the main body cavity of the main body.

3. The fecal collection apparatus of claim 2, wherein the plunger defines a sample collection aperture defining an aperture inner diameter, the sample collection member insertable through the sample collection aperture.

4. The fecal collection apparatus of claim 3, wherein the fecal engagement portion is deformable between a compressed position and an uncompressed position, the fecal engagement portion defining an uncompressed sample collection outer diameter when in the uncompressed position that is greater than the aperture inner diameter, and a compressed sample collection outer diameter when in the compressed position that is less than the uncompressed sample collection outer diameter.

5. The fecal collection apparatus of claim 2, further comprising a guide cap positioned at the insertion end of the main body.

6. The fecal collection apparatus of claim 5, wherein:
the guide cap comprises a plurality of teeth;
the main body includes a flange extending from the insertion end; and
the flange of the main body engages the plurality of teeth of the guide cap to retain the guide cap on the main body.

7. The fecal collection apparatus of claim 5, wherein the guide cap defines a rail and the plunger comprises a slotted portion, the plunger is rotatable relative to the guide cap to position the rail of the guide cap within the slotted portion of the plunger.

8. The fecal collection apparatus of claim 7, wherein the plunger is inhibited from moving in an axial direction when the rail of the guide cap is positioned within the slotted portion of the plunger, and wherein the plunger is permitted to move in the axial direction when the rail of the guide cap is not positioned within the slotted portion of the plunger.

9. The fecal collection apparatus of claim 1, further comprising a brush positioned within the main body cavity of the main body proximate the removal end, the brush having a plurality of bristles for engaging the fecal engagement portion of the sample collection member.

10. The fecal collection apparatus of claim 1, further comprising an emulsifying buffer positioned in the main body cavity.

11. The fecal collection apparatus of claim 1, wherein the plug is coupled to the main body through a frangible seal.

12. The fecal collection apparatus of claim 1, wherein the plug is monolithic with the main body.

13. A fecal collection apparatus comprising:
a main body comprising an insertion end and a removal end;
a plunger positioned at least partially within the main body;
a sample collection member positionable within the main body through the insertion end, the sample collection member defining a fecal engagement portion for holding a fecal sample; and
a guide cap positioned at the insertion end of the main body,
wherein:
the plunger is movable with respect to the guide cap between a locked state and an unlocked state;
the plunger is inhibited from moving in an axial direction with the plunger in the locked state, the axial direction corresponding to a longitudinal axis extending from the insertion end to the removal end of the main body; and
the plunger is moveable in the axial direction with the plunger in the unlocked state.

14. The fecal collection apparatus of claim 13, wherein the plunger defines a sample collection aperture defining an aperture inner diameter.

15. The fecal collection apparatus of claim 14, wherein the sample collection member comprises a handle portion and the fecal engagement portion opposite the handle portion, the fecal engagement portion being deformable between a compressed position and an uncompressed position, the fecal engagement portion defining an uncompressed sample collection outer diameter that is greater than the aperture inner diameter when in the uncompressed position.

16. The fecal collection apparatus of claim 13, wherein:
the guide cap comprises a plurality of teeth; and
the main body includes a flange extending from the insertion end, wherein the flange of the main body engages the plurality of teeth of the guide cap to retain the guide cap on the main body.

17. The fecal collection apparatus of claim 13, wherein the guide cap defines a rail and the plunger comprises a slotted portion, the plunger is rotatable relative to the guide cap to position the rail of the guide cap within the slotted portion of the plunger.

18. The fecal collection apparatus of claim 13, further comprising a brush positioned within the main body, the brush having a plurality of bristles for engaging the fecal engagement portion of the sample collection member.

19. A fecal collection attachment device positionable onto a syringe, the fecal collection attachment device comprising:
a guide cap positionable onto the syringe, the guide cap defining a rail;
a plunger comprising a slotted portion, the plunger rotatable relative to the guide cap to position the rail of the guide cap within the slotted portion of the plunger; and
a sample collection member positionable through the plunger, the sample collection member defining a fecal engagement portion for holding a fecal sample,
wherein the plunger is inhibited from moving in an axial direction relative to the guide cap when the rail of the guide cap is positioned within the slotted portion of the plunger, and wherein the plunger is permitted to move in the axial direction when the rail of the guide cap is not positioned within the slotted portion of the plunger.

20. The fecal collection attachment device of claim 19, wherein the plunger defines a sample collection aperture defining an aperture inner diameter, and wherein the sample collection member comprises a handle portion and the fecal engagement portion opposite the handle portion, the fecal engagement portion being deformable between a compressed position and an uncompressed position, the collection end defining an uncompressed sample collection outer diameter that is greater than the aperture inner diameter when in the uncompressed position.
